(19) United States
(12) Patent Application Publication
(10) Pub. No.: US 2011/0123482 A1
(43) Pub. Date: May 26, 2011

(54) Methods of Treating Neurological Autoimmune Disorders with Cyclophosphamide

(75) Inventors: Adam Ian Kaplin, Baltimore, MD (US); Douglas Kerr, Ruxton, MD (US); Peter Arthur Calabresi, Lutherville, MD (US); Sithy Rameeza Allie, Lebanon, NH (US)

(73) Assignee: The Johns Hopkins University, Baltimore, MD (US)

(21) Appl. No.: 12/681,237

(22) PCT Filed: Oct. 1, 2008
(86) PCT No.: PCT/US08/11402

§ 371 Date: Jan. 18, 2011

Related U.S. Application Data
(60) Provisional application No. 60/997,074, filed on Oct. 1, 2007.
(60) Provisional application No. 61/037,059, filed on Mar. 17, 2008.
(60) Provisional application No. 61/083,607, filed on Jul. 25, 2008.

(51) Int. Cl. A61K 38/19; C12Q 1/32; C07F 9/6584; A61K 31/675; A61P 25/00; A61P 25/18

(52) U.S. Cl. 424/85.1; 435/26; 564/13; 514/110

(57) ABSTRACT

Described herein are methods for treating neurological autoimmune disorders in which the treatment method includes administering an immunoablative agent to eliminate most or essentially all maturing and mature elements of the immune system in an affected individual. Following this step, the individual is administered agents to reestablish the ablated immune system.

### CROSS-REFERENCE

This application claims the benefit of U.S. Provisional Application No. 61/083,607, filed Jul. 25, 2008, U.S. Provisional Application No. 61/037,059, filed Mar. 17, 2008, and U.S. Provisional Application No. 60/997,074, filed Oct. 1, 2007, which are each incorporated herein by reference in their entirety.

### BACKGROUND OF THE INVENTION

Autoimmune disorders are disorders characterized by an immune system's failure to recognize self. Examples of neurological autoimmune disorders include, but are not limited to, multiple sclerosis, Guillain-Barre syndrome, Lambert-Eaton myasthenic syndrome, myasthenia gravis, transverse myelitis, systemic lupus erythematosus (SLE or lupus), acute disseminated encephalomyelitis, autoimmune inner ear disease, narcolepsy, neuromyotonia, and schizophrenia. Multiple Sclerosis is an autoimmune disease characterized by a demyelination and axonal injury of neurons and gliosis. It affects between about 2 and 150 people per 100,000.

### SUMMARY OF THE INVENTION

In some embodiments, the treatment method comprises administering an agent that acts as an immunoablative agent to an individual having a neurological autoimmune disorder. In some embodiments, the immunoablative agent is a chemotherapeutic agent, a biologic, or a radioactive agent. In some embodiments, the chemotherapeutic agent is a cytostatic, an alkylating agent, an anti-metabolite, and cytotoxic antibiotics. In some embodiments, the alkylating agent is an oxazophorine. In some embodiments, the oxazophorine is cyclophosphamide. In some embodiments, the biologic is a T cell depleting antibody. In some embodiments, the T cell depleting antibody is antilymphocyte globulin, antithymocyte globulin (ATG), an anti-IL-2 receptor antibody, an anti-CD 3 receptor antibody (e.g. OKT3), or combinations thereof.

In further embodiments, after the individual has received a therapeutically-effective amount of the immunoablative agent, the individual is administered a therapeutically-effective amount of at least one immune system reconstituting agent to reconstitute the individual's ablated immune system. In some embodiments, the one immune system reconstituting agent is a colony stimulating factor, hematopoietic stem cells, or combinations thereof. In some embodiments, the colony stimulating factor is granulocyte-macrophage CSF (GM-CSF), granulocyte CSF (G-CSF) and macrophage CSF (M-CSF), or combinations thereof.

In some embodiments, after the individual has received (a) a therapeutically-effective amount of the immunoablative agent, and (b) a therapeutically-effective amount of at least one immune system reconstituting agent, the individual is administered a therapeutically-effective amount of at least one immunomodulatory agent. In some embodiments, the immunomodulatory agent is glatiramer acetate, 5-aminoimidazole-4-carboxamide ribonucleoside (AICAR), an interferon (e.g. IFNβ-1a, and IFNβ-1b), or combinations thereof.

Disclosed herein, in certain embodiments, is a method of treating neurological autoimmune disorders, comprising administering to an individual in need having an aldehyde dehydrogenase level in the CD 4+ T cells less than a predetermined threshold: (a) about 10 to about 70 mg/kg/day of cyclophosphamide; (b) about 1 to about 10 μg/kg/day of granulocyte colony stimulating factor; and (c) about 10 mg/day to about 80 mg/day of glatiramer acetate. In some embodiments, the method further comprises determining the level of aldehyde dehydrogenase in the individual's CD 4+ T cells. In some embodiments, the method further comprises monitoring the level of aldehyde dehydrogenase in the individual's CD 4+ T cells. In some embodiments, at least about 50 mg/kg/day of cyclophosphamide is administered to the individual. In some embodiments, at least about 5 μg/kg/day of granulocyte colony stimulating factor is administered to the individual. In some embodiments, at least about 40 mg/day of glatiramer acetate is administered to the individual. In some embodiments, the method further comprises controlling access to the treatment using a method that comprises a first screen, a second screen, and restricted distribution of the cyclophosphamide. In some embodiments, the first screen comprises: (a) determining whether the individual complies with treatment criteria; (b) if the individual is female, testing the individual for pregnancy and providing the individual with pregnancy counseling; (c) determining the level of aldehyde dehydrogenase associated with the individual's CD 4+ T cells; and (d) matching the individual with a supply of red blood cells and platelets. In some embodiments, the second screen comprises monitoring the individual for pregnancy, if the individual is female; and/or adverse events. In some embodiments, the adverse event is toxicity. In some embodiments, an individual is removed from treatment if the individual is pregnant, and/or experiences an adverse event. In some embodiments, the restricted distribution of the cyclophosphamide comprises: (a) assigning each individual an identification number; (b) associating an identification number with a container of cyclophosphamide; and (c) administering cyclophosphamide from the container of cyclophosphamide to an individual whose identification number corresponds to the identification number associated with the container. In some embodiments, the cyclophosphamide is administered for at least about four consecutive days. In some embodiments, administration of the granulocyte colony stimulating factor is initiated within five to seven days after administration of the cyclophosphamide has been completed. In some embodiments, the granulocyte colony stimulating factor is administered to the individual until the individual's absolute neutrophil count exceeds about 1.0×10⁹ cells/L for two consecutive days. In some embodiments, administration of the glatiramer acetate is initiated within 28 to 35 days after administration of the cyclophosphamide has been completed. In some embodiments, the dose of glatiramer acetate is at least about 40 mg/day. In some embodiments, within 2.5 to 4 months after the dose of glatiramer acetate is initiated, the dose of glatiramer acetate is reduced to about 20 mg/day. In some embodiments, the autoimmune neurological disorder is multiple sclerosis, Guillain-Barre syndrome, Lambert-Eaton myasthenic syndrome, myasthenia gravis, transverse myelitis, systemic lupus erythematosus (SLE or lupus), acute disseminated encephalomyelitis, autoimmune inner ear disease, narcolepsy, neuromyotonia, schizophrenia, or combinations thereof. In some embodiments, the autoimmune neurological disorder is multiple sclerosis. In some embodiments, the multiple sclerosis has relapsed. In some embodiments, the multiple sclerosis is in remission. In some embodiments, the cyclophosphamide is prepared from reconstituted lyophilized cyclophosphamide. In some embodiments, the cyclophosphamide is administered intravenously. In some embodiments, the method further comprises administering to the individual up to about 5 mg/kg/day of antithymocyte globulin.

Disclosed herein, in certain embodiments, is a method of treating neurological autoimmune disorders, comprising administering to an individual in need having an aldehyde dehydrogenase level in the CD 4+ T cells less than a predetermined threshold: (a) about 10 to about 70 mg/kg/day of cyclophosphamide; (b) up to about 5 mg/kg/day of antithymocyte globulin; and (c) about 1 to about 10 μg/kg/day of granulocyte colony stimulating factor. In some embodiments, the method further comprises determining the level of aldehyde dehydrogenase in the individual's CD 4+ T cells. In some embodiments, the method further comprises monitoring the level of aldehyde dehydrogenase in the individual's CD 4+ T cells. In some embodiments, at least about 50 mg/kg/day of cyclophosphamide is administered to the individual. In some embodiments, at least about 5 μg/kg/day of granulocyte colony stimulating factor is administered to the individual. In some embodiments, at least about 2.5 μg/kg/day of antithymocyte globulin is administered to the individual. In some embodiments, the method further comprises controlling access to the treatment using a method that comprises a first screen, a second screen, and restricted distribution of the cyclophosphamide. In some embodiments, the first screen comprises: (a) determining whether the individual complies with treatment criteria; (b) if the individual is female, testing the individual for pregnancy and providing the individual with pregnancy counseling; (c) determining the level of aldehyde dehydrogenase associated with the individual's CD 4+ T cells; and (d) matching the individual with a supply of red blood cells and platelets. In some embodiments, the second screen comprises monitoring the individual for pregnancy, if the individual is female; and/or adverse events. In some embodiments, the adverse event is toxicity. In some embodiments, an individual is removed from treatment if the individual is pregnant, and/or experiences an adverse event. In some embodiments, the restricted distribution of the cyclophosphamide comprises: (a) assigning each individual an identification number; (b) associating an identification number with a container of cyclophosphamide; and (c) administering cyclophosphamide from the container of cyclophosphamide to an individual whose identification number corresponds to the identification number associated with the container. In some embodiments, the cyclophosphamide is administered for at least about four consecutive days. In some embodiments, the antithymocyte globulin is administered before, after, or simultaneously with the cyclophosphamide. In some embodiments, administration of the granulocyte colony stimulating factor is initiated within five to seven days after administration of the cyclophosphamide has been completed. In some embodiments, the granulocyte colony stimulating factor is administered to the individual until the individual's absolute neutrophil count exceeds about 1.0×10⁹ cells/L for two consecutive days. In some embodiments, the autoimmune neurological disorder is multiple sclerosis, Guillain-Barre syndrome, Lambert-Eaton myasthenic syndrome, myasthenia gravis, transverse myelitis, systemic lupus erythematosus (SLE or lupus), acute disseminated encephalomyelitis, autoimmune inner ear disease, narcolepsy, neuromyotonia, schizophrenia, or combinations thereof. In some embodiments, the autoimmune neurological disorder is multiple sclerosis. In some embodiments, the multiple sclerosis has relapsed. In some embodiments, the multiple sclerosis is in remission. In some embodiments, the cyclophosphamide is prepared from reconstituted lyophilized cyclophosphamide. In some embodiments, the cyclophosphamide is administered intravenously. In some embodiments, the method further comprises administering to the individual about 10 mg/day to about 80 mg/day of glatiramer acetate.

Disclosed herein, in certain embodiments, is a method of selecting an individual for treatment with cyclophosphamide comprising selecting an individual for treatment if an aldehyde dehydrogenase level in a biological sample from the individual exceeds a predetermined threshold; or selecting an alternative treatment if the aldehyde dehydrogenase level observed in the biological sample is below a predetermined threshold. In some embodiments, the biological sample is blood, and/or white blood cells. In some embodiments, the white blood cells are T cells. In some embodiments, the T cells are CD 4+ T cells. In some embodiments, the aldehyde dehydrogenase level is determined by a fluorescent aldehyde dehydrogenase substrate assay. In some embodiments, the fluorescent aldehyde dehydrogenase substrate is ALDEFLUOR®. In some embodiments, the aldehyde dehydrogenase level is determined by measuring RNA levels. In some embodiments, the aldehyde dehydrogenase level is measured by contacting the biological sample with antibodies to aldehyde dehydrogenase. In some embodiments, the antibody is isotopically-labeled, radio-labeled, fluorophore-labeled, or biotinylated. In some embodiments, the selected individual is administered cyclophosphamide. In some embodiments, the individual has an autoimmune neurological disorder selected from multiple sclerosis, Guillain-Barre syndrome, Lambert-Eaton myasthenic syndrome, myasthenia gravis, transverse myelitis, systemic lupus erythematosus (SLE or lupus), acute disseminated encephalomyelitis, autoimmune inner ear disease, narcolepsy, neuromyotonia, schizophrenia, or combinations thereof. In some embodiments, the autoimmune neurological disorder is multiple sclerosis.

Disclosed herein, in certain embodiments, is a method of monitoring an individual being administered cyclophosphamide, comprising determining the level of aldehyde dehydrogenase in at least a first biological sample and a second biological sample, wherein the first biological sample and the second biological sample are taken from the individual at different times. In some embodiments, the method further comprises discontinuing treatment if a level of aldehyde dehydrogenase observed in a biological sample exceeds a predetermined threshold. In some embodiments, the method further comprises altering treatment based on the level of aldehyde dehydrogenase observed in a first biological sample, the second biological sample, or a combination thereof. In some embodiments, the method further comprises selecting an alternative treatment if the level of aldehyde dehydrogenase observed in the first biological sample, the second biological sample, or a combination thereof exceeds a predetermined threshold. In some embodiments, the biological sample is blood, and/or white blood cells. In some embodiments, the white blood cells are T cells. In some embodiments, the T cells are CD 4+ T cells. In some embodiments, the level of aldehyde dehydrogenase is determined by a fluorescent aldehyde dehydrogenase substrate assay. In some embodiments, the fluorescent aldehyde dehydrogenase substrate is ALDEFLUOR®. In some embodiments, the level of aldehyde dehydrogenase is determined by measuring RNA levels. In some embodiments, the level of aldehyde dehydrogenase is determined by contacting the biological sample with antibodies to aldehyde dehydrogenase. In some embodiments, the antibody is isotopically-labeled, radio-labeled, fluorophore-labeled, or biotinylated. In some embodiments, the individual has an autoimmune neurological disorder selected from multiple sclerosis, Guillain-Barre syndrome, Lambert-Eaton myasthenic syndrome, myasthenia gravis, transverse myelitis, systemic lupus erythematosus (SLE or lupus), acute disseminated encephalomyelitis, autoimmune inner ear disease, narcolepsy, neuromyotonia, schizophrenia, or combinations thereof. In some embodiments, the autoimmune neurological disorder is multiple sclerosis.

Disclosed herein, in certain embodiments, is a method of selecting an individual for treatment with cyclophosphamide comprising contacting a biological sample from the individual with cyclophosphamide. In some embodiments, the method further comprises determining the level of cell death in the sample after contacting the biological sample from the individual with cyclophosphamide. In some embodiments, the biological sample is blood, and/or white blood cells. In some embodiments, the white blood cells are T cells. In some embodiments, the T cells are CD 4+ T cells. In some embodiments, the level of aldehyde dehydrogenase is determined by a fluorescent aldehyde dehydrogenase substrate assay. In some embodiments, the fluorescent aldehyde dehydrogenase substrate is ALDEFLUOR®. In some embodiments, the level of aldehyde dehydrogenase is determined by measuring RNA levels. In some embodiments, the level of aldehyde dehydrogenase is determined by contacting the biological sample with antibodies to aldehyde dehydrogenase. In some embodiments, the antibody is isotopically-labeled, radio-labeled, fluorophore-labeled, or biotinylated. In some embodiments, the individual has an autoimmune neurological disorder selected from multiple sclerosis, Guillain-Barre syndrome, Lambert-Eaton myasthenic syndrome, myasthenia gravis, transverse myelitis, systemic lupus erythematosus (SLE or lupus), acute disseminated encephalomyelitis, autoimmune inner ear disease, narcolepsy, neuromyotonia, schizophrenia, or combinations thereof. In some embodiments, the autoimmune neurological disorder is multiple sclerosis.

Disclosed herein, in certain embodiments, is method of monitoring an individual being administered cyclophosphamide, comprising contacting a biological sample from the individual with cyclophosphamide. In some embodiments, the method further comprises determining the level of cell death in the sample after contacting the biological sample from the individual with cyclophosphamide. In some embodiments, the method further comprises discontinuing treatment if the level of cell death observed in the biological sample is below a predetermined threshold. In some embodiments, the method further comprises altering treatment based on the level of cell death observed in the biological sample. In some embodiments, the method further comprises selecting an alternative treatment if the level of cell death observed in the biological sample is below a predetermined threshold. In some embodiments, the biological sample is blood, and/or white blood cells. In some embodiments, the white blood cells are T cells. In some embodiments, the T cells are CD 4+ T cells. In some embodiments, the level of aldehyde dehydrogenase is determined by a fluorescent aldehyde dehydrogenase substrate assay. In some embodiments, the fluorescent aldehyde dehydrogenase substrate is ALDEFLUOR®. In some embodiments, the level of aldehyde dehydrogenase is determined by measuring RNA levels. In some embodiments, the level of aldehyde dehydrogenase is determined by contacting the biological sample with antibodies to aldehyde dehydrogenase. In some embodiments, the antibody is isotopically-labeled, radio-labeled, fluorophore-labeled, or biotinylated. In some embodiments, the individual has an autoimmune neurological disorder selected from multiple sclerosis, Guillain-Barre syndrome, Lambert-Eaton myasthenic syndrome, myasthenia gravis, transverse myelitis, systemic lupus erythematosus (SLE or lupus), acute disseminated encephalomyelitis, autoimmune inner ear disease, narcolepsy, neuromyotonia, schizophrenia, or combinations thereof. In some embodiments, the autoimmune neurological disorder is multiple sclerosis.

Disclosed herein, in certain embodiments, is a composition, comprising cyclophosphamide in solution, wherein the cyclophosphamide in solution has been reconstituted from lyophilized cyclophosphamide. In some embodiments, the cyclophosphamide is reconstituted in phosphate buffered saline. In some embodiments, the concentration of cyclophosphamide in the solution is at least about 20 mg/ml. In some embodiments, the composition is for use as an immunoablative agent in an individual with an autoimmune neurological disorder. In some embodiments, the individual has an autoimmune neurological disorder selected from multiple sclerosis, Guillain-Barre syndrome, Lambert-Eaton myasthenic syndrome, myasthenia gravis, transverse myelitis, systemic lupus erythematosus (SLE or lupus), acute disseminated encephalomyelitis, autoimmune inner ear disease, narcolepsy, neuromyotonia, schizophrenia, or combinations thereof. In some embodiments, the autoimmune neurological disorder is multiple sclerosis.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is an illustrative graphical representation showing that no individuals treated with HiGa have experienced reactivation of their MS in the time that they have been followed.

### DETAILED DESCRIPTION OF THE INVENTION

In some embodiments, the treatment method comprises administering an agent that acts as an immunoablative agent to an individual having a neurological autoimmune disorder. In some embodiments, the immunoablative agent is a chemotherapeutic agent, a biologic, or a radioactive agent. In some embodiments, the chemotherapeutic agent is a cytostatic, an alkylating agent, an anti-metabolite, and cytotoxic antibiotics. In some embodiments, the alkylating agent is an oxazophorine. In some embodiments, the oxazophorine is cyclophosphamide. In some embodiments, the biologic is a T cell depleting antibody. In some embodiments, the T cell depleting antibody is antilymphocyte globulin, antithymocyte globulin (ATG), an anti-IL-2 receptor antibody, an anti-CD 3 receptor antibody (e.g. OKT3), or combinations thereof.

In further embodiments, after the individual has received a therapeutically-effective amount of the immunoablative agent, the individual is administered a therapeutically-effective amount of at least one immune system reconstituting agent to reconstitute the individual's ablated immune system. In some embodiments, the one immune system reconstituting agent is a colony stimulating factor, hematopoietic stem cells, or combinations thereof. In some embodiments, the colony stimulating factor is granulocyte-macrophage CSF (GM-CSF), granulocyte CSF (G-CSF) and macrophage CSF (M-CSF), or combinations thereof.

In some embodiments, after the individual has received (a) a therapeutically-effective amount of the immunoablative agent, and (b) a therapeutically-effective amount of at least one immune system reconstituting agent, the individual is administered a therapeutically-effective amount of at least one immunomodulatory agent. In some embodiments, the immunomodulatory agent is glatiramer acetate, 5-aminoimidazole-4-carboxamide ribonucleoside (AICAR), an interferon (e.g. IFNβ-1a, and IFNβ-1b), or combinations thereof.

### CERTAIN DEFINITIONS

Unless indicated otherwise, the following terms have the following meanings when used herein and in the appended claims.

The term “lymphocyte” encompasses, by way of non-limiting example, B-cells, T-cells, NKT cells, and NK cells. In some embodiments lymphocytes refers to immature, mature, undifferentiated and differentiated white lymphocyte populations including tissue specific and specialized varieties. In some embodiments lymphocytes include B-cell lineages including pre-B-cells, Progenitor B cells, Early Pro-B cells, Late Pro-B cells, Large Pre-B cells, Small Pre-B cells, Immature B cells, Mature B cells, plasma B-cells, memory B-cells, B-1 cells, B-2 cells and anergic AN1/T3 cell populations.

The term B-cell, refers to, by way of non-limiting example, a pre-B-cell, Progenitor B cell, Early Pro-B cell, Late Pro-B cell, Large Pre-B cell, Small Pre-B cell, Immature B cell, Mature B cell, plasma B-cell, memory B-cell, B-1 cell, B-2 cells and anergic AN1/T3 cell populations. In some embodiments the term B-cell includes a B-cell that expresses an immunoglobulin heavy chain and/or light chain on its cells surface. In some embodiments the term B-cell includes a B-cell that expresses and secretes an immunoglobulin heavy chain and/or light chain. In some embodiments the term B-cell includes a cell that binds an antigen on its cell-surface. In some embodiments disclosed herein, B-cells or AN1/T3 cells are utilized in the processes described. In certain embodiments, such cells are optionally substituted with any animal cell suitable for expressing, capable of expressing (e.g., inducible expression), or capable of being differentiated into a cell suitable for expressing an antibody including, e.g., a hematopoietic stem cell, a B-cell, a pre-B-cell, a Progenitor B cell, a Early Pro-B cell, a Late Pro-B cell, a Large Pre-B cell, a Small Pre-B cell, an Immature B cell, a Mature B cell, a plasma B-cell, a memory B-cell, a B-1 cell, a B-2 cell, an anergic B-cell, or an anergic AN1/T3 cell.

The term “antigen” refers to a substance that is capable of inducing the production of an antibody. In some embodiments an antigen is a substance that binds to an antibody variable region.

The term “expression” refers to one or more of the following events: (1) production of an RNA template from a DNA sequence (e.g., by transcription) within a cell; (2) processing of an RNA transcript (e.g., by splicing, editing, 5′ cap formation, and/or 3′ end formation) within a cell; (3) translation of an RNA sequence into a polypeptide or protein within a cell; (4) post-translational modification of a polypeptide or protein within a cell; (5) presentation of a polypeptide or protein on the cell surface; (6) secretion or release of a polypeptide or protein from a cell.

The terms “antibody” and “antibodies” refer to monoclonal antibodies, polyclonal antibodies, bi-specific antibodies, multispecific antibodies, grafted antibodies, human antibodies, humanized antibodies, synthetic antibodies, chimeric antibodies, camelized antibodies, single-chain Fvs (scFv), single chain antibodies, Fab fragments, F(ab′) fragments, disulfide-linked Fvs (sdFv), intrabodies, and anti-idiotypic (anti-Id) antibodies and antigen-binding fragments of any of the above. In particular, antibodies include immunoglobulin molecules and immunologically active fragments of immunoglobulin molecules, i.e., molecules that contain an antigen binding site. Immunoglobulin molecules are of any type (e.g., IgG, IgE, IgM, IgD, IgA and IgY), class (e.g., IgG₁, IgG₂, IgG₃, IgG₄, IgA₁ and IgA₂) or subclass. The terms “antibody” and immunoglobulin are used interchangeably in the broadest sense. The subunit structures and three-dimensional configurations of the different classes of immunoglobulins are well known in the art. In some embodiments an antibody is part of a larger molecule, formed by covalent or non-covalent association of the antibody with one or more other proteins or peptides.

The antibodies herein include monoclonal, polyclonal, recombinant, chimeric, humanized, bi-specific, grafted, human, and fragments thereof including antibodies altered by any means to be less immunogenic in humans. Thus, for example, the monoclonal antibodies and fragments, etc., herein include “chimeric” antibodies and “humanized” antibodies. In general, chimeric antibodies include a portion of the heavy and/or light chain that is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, so long as they exhibit the desired biological activity (U.S. Pat. No. 4,816,567); Morrison et al. Proc. Natl. Acad. Sci. 81:6851-6855 (1984). For example in some embodiments a chimeric antibody contains variable regions derived from a mouse and constant regions derived from human in which the constant region contains sequences homologous to both human IgG2 and human IgG4. Numerous methods for preparing “chimeric” antibodies, etc., are known in the art. “Humanized” forms of non-human (e.g., murine) antibodies or fragments are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include, grafted antibodies or CDR grafted antibodies wherein part or all of the amino acid sequence of one or more complementarily determining regions (CDRs) derived from a non-human animal antibody is grafted to an appropriate position of a human antibody while maintaining the desired binding specificity and/or affinity of the original non-human antibody. In some embodiments, corresponding non-human residues replace Fv framework residues of the human immunoglobulin. In some embodiments humanized antibodies comprise residues that are found neither in the recipient antibody nor in the imported CDR or framework sequences. These modifications are made to further refine and optimize antibody performance. In some embodiments, the humanized antibody comprises substantially all of at least one, and typically two, variable domains, in which all or substantially all of the CDR regions correspond to those of a non-human immunoglobulin and all or substantially all of the FR regions are those of a human immunoglobulin consensus sequence. For further details, see, e.g.: Jones et al., Nature 321: 522-525 (1986); Reichmann et al., Nature 332: 323-329 (1988) and Presta, Curr. Op. Struct. Biol. 2: 593-596 (1992). Numerous methods for “humanizing” antibodies, etc., are known in the art.

The terms “polypeptide”, peptide” and “protein” are used interchangeably herein to refer to a polymer of amino acid residues. The terms apply to naturally occurring amino acid polymers as well as amino acid polymers in which one or more amino acid residues is a non-naturally occurring amino acid, e.g., an amino acid analog. The terms encompass amino acid chains of any length, including full length proteins, wherein the amino acid residues are linked by covalent peptide bonds.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrolysine and selenocysteine. Amino acid analogs refers to agents that have the same basic chemical structure as a naturally occurring amino acid, i.e., an α carbon that is bound to a hydrogen, a carboxyl group, an amino group, and an R group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid.

Amino acids are referred to herein by either their commonly known three letter symbols or by the one-letter symbols recommended by the IUPAC-IUB Biochemical Nomenclature Commission. Nucleotides, likewise, are referred to by their commonly accepted single-letter codes.

The term “nucleic acid” refers to deoxyribonucleotides, deoxyribonucleosides, ribonucleosides, or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless specifically limited otherwise, the term also refers to oligonucleotide analogs including PNA (peptidonucleic acid), analogs of DNA used in antisense technology (phosphorothioates, phosphoroamidates, and the like). Unless otherwise indicated, a particular nucleic acid sequence also implicitly encompasses conservatively modified variants thereof (including but not limited to, degenerate codon substitutions) and complementary sequences as well as the sequence explicitly indicated. Specifically, degenerate codon substitutions are achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Cassol et al. (1992); Rossolini et al., Mol. Cell. Probes 8:91-98 (1994)).

The terms “treat”, “treatment”, and “treating” refer to include: alleviating, abating or ameliorating a disease or condition (e.g. MS, Guillain-Barre syndrome, Lambert-Eaton myasthenic syndrome, myasthenia gravis, or transverse myelitis), as well as symptoms of the disease or condition; preventing additional symptoms; ameliorating or preventing the underlying metabolic causes of symptoms; inhibiting the disease or condition, e.g., arresting the development of the disease or condition; relieving the disease or condition; causing regression of the disease or condition; relieving a condition caused by the disease or condition; or stopping the symptoms of the disease or condition either prophylactically and/or therapeutically.

The term “prodrug” refers to a compound or agent that is converted into an active form in vivo. In certain embodiments, a prodrug is enzymatically metabolized by one or more steps or processes to the biologically, pharmaceutically or therapeutically active form of the compound. To produce a prodrug, a pharmaceutically active compound is modified such that the active compound will be regenerated upon in vivo administration. In one embodiment, the prodrug is designed to alter the metabolic stability or the transport characteristics of a drug, to mask side effects or toxicity, or to alter other characteristics or properties of a drug.

The term “individual” is used to mean an animal, preferably a mammal, including a human or non-human. The terms individual, subject, and patient may be used interchangeably. None of the terms require that the individual be under the care of a medical professional (e.g. a physician, nurse, hospice care worker, orderly, or physician's assistant).

The terms “effective amount” or “therapeutically effective amount,” refer to a sufficient amount of the agents disclosed herein being administered that would be expected to relieve to some extent one or more of the symptoms of the disease or condition being treated. For example, the result of administration of cyclophosphamide is a reduction and/or complete elimination of mature and/or maturing cells (e.g. lymphoid cells, natural killer cells, B-, and T-lymphocytes). The term “therapeutically effective amount” includes, for example, a prophylactically effective amount. An “effective amount” of an agent disclosed herein is an amount effective to achieve a desired pharmacologic effect or therapeutic improvement without undue adverse side effects. It is understood that “an effective amount” or “a therapeutically effective amount” varies, in some embodiments, from individual to individual, due to variation in metabolism of the compound administered, age, weight, general condition of the individual, the condition being treated, the severity of the condition being treated, and the judgment of the prescribing physician. It is also understood that “an effective amount” in an extended-release dosing format may differ from “an effective amount” in an immediate-release dosing format based upon pharmacokinetic and pharmacodynamic considerations

### Neurological Autoimmune Disorders

In some embodiments, an individual is being treated for an autoimmune disorder (e.g. the individual has been diagnosed with an autoimmune disorder, the individual is suspected of having an autoimmune disorder, or the individual is predisposed to develop an autoimmune disorder). In certain instances, an autoimmune disorder is characterized by an immune system's attacking self (e.g. its own cells). In some embodiments, the autoimmune disorder is a neurological autoimmune disorder (e.g. an immune system attacking most, essentially all, or part of the Peripheral Nervous System, most, essentially all, or part of the Central Nervous System, a nerve, a neuron, and myelin). In some embodiments, the neurological autoimmune disorder is multiple sclerosis, Guillain-Barre syndrome, Lambert-Eaton myasthenic syndrome, myasthenia gravis, transverse myelitis, systemic lupus erythematosus (SLE or lupus), acute disseminated encephalomyelitis, autoimmune inner ear disease, narcolepsy, neuromyotonia, schizophrenia, or combinations thereof.

Multiple Sclerosis

In some embodiments, the neurological autoimmune disorder is multiple sclerosis (also known as MS, disseminated sclerosis, or encephalomyelitis disseminata). MS is an autoimmune disease that, in certain circumstances, is characterized by recurrent episodes of demyelination and inflammation within the central nervous system. In certain instances, the demyelineation of a neuron results in a neuron with a compromised ability to conduct electrical signals.

Symptoms of MS include, but are not limited to, changes in sensation (e.g. hypoesthesias and paraesthesias), muscle weakness, muscle spasms, difficulty moving; difficulty with coordination and/or balance (e.g. ataxia); difficulty speaking (e.g. dysarthria), difficulty swallowing (e.g. dysphagia), difficulty controlling eye movement (e.g. nystagmus), impaired vision (e.g. diplopia), fatigue, pain (e.g. acute or chronic), difficulty controlling bladder function, difficulty controlling bowel function, and depression.

MS commonly presents as relapsing-remitting (RRMS). Relapsing-remitting MS is comprised of periods of relapse/exacerbation (e.g. the unprovoked and unanticipated occurrence of a new symptom, or recurrence of an old symptom, lasting for a period of greater than 24 hours) followed by periods of remission (e.g. periods with limited or no MS symptoms). In about 50% of individuals diagnosed with RRMS, the disorder progresses to secondary progressive MS. Secondary progressive MS (SPMS) is characterized by an initial period of relapsing-remitting MS, followed by progressive neurologic decline between relapses without any definite periods of remission. In some embodiments, the multiple sclerosis has relapsed. In some embodiments, the multiple sclerosis is in remission. In some embodiments, the multiple sclerosis in a progressive phase.

MS is often diagnosed using the McDonald Criteria. Table 1 sets forth the additional data needed to diagnose MS based on an individual's clinical presentation.

TABLE 1

| Clinical Presentation | Additional Data Needed |
|---|---|
| 2 or more attacks | None; clinical evidence will suffice |
| (relapses) | (additional evidence desirable but must be |
| 2 or more objective | consistent with MS) |
| clinical lesions | |
| 2 or more attacks | Dissemination in space, demonstrated by: |
| 1 objective clinical | MRI |
| lesion | or a positive CSF and 2 or more |
| | MRI lesions consistent with MS |
| | or further clinical attack involving |
| | different site |
| 1 attack | Dissemination in time, demonstrated by: |
| 2 or more objective | MRI |
| clinical lesions | or second clinical attack |
| 1 attack | Dissemination in space by demonstrated |
| 1 objective clinical | by: |
| lesion | MRI |
| (monosymptomatic | or positive CSF and 2 or more MRI |
| presentation) | lesions consistent with MS |
| | and |
| | Dissemination in time demonstrated by: |
| | MRI |
| | or second clinical attack |
| Insidious neurological | Positive CSF |
| progression suggestive of | and |
| MS (primary progressive | Dissemination in space demonstrated by: |
| MS) | MRI evidence of 9 or more T2 |
| | brain lesions |
| | or 2 or more spinal cord lesions |
| | or 4-8 brain and 1 spinal cord lesion |
| | or positive VHP with 4-8 MRI |
| | lesions |
| | or positive VEP with <4 brain |
| | lesions plus 1 spinal cord lesion |
| | and |
| | Dissemination in time demonstrated by: |
| | MRI |
| | or continued progression for 1 year |

The main clinical measure of disability progression and symptom severity is the Expanded Disability Status Scale or EDSS. A commonly used clinical rating scale, the EDSS ranges from 0 (normal) to 10 (death due to MS), based on neurological examination of eight functional systems (visual, brainstem, sensory, cerebellar, sphincter, cerebral and others). It is a useful tool for classifying MS individuals by disease severity. It measures impairment and disability based on the ratings of an observer or neurologist through a structured interview.

An additional measure of disability progression and symptom severity is the Multiple Sclerosis Functional Composite (MSFC). This scale is based on the composite score of three individual tests designed to test gait, upper extremity dexterity and cognition. The three subtests are: a) 25 foot timed walk (25TW); b) 9-hole peg test (9-HPT); and c) Paced Auditory Serial Addition Test (PASAT-3). The PASAT test requires individuals to add consecutive numbers as they are presented on an auditory tape and respond orally with the accurate sum. As each digit is presented, the individual must sum that number with the digit that was presented prior to it rather than with the individual's previous response.

By way of non-limiting example, MS symptoms are treated with corticosteroids (e.g. 500 to 1,000 mg of intravenous methylprednisolone followed by a tapering dose of oral prednisone over several weeks), interferons (e.g. IFNβ-1a, and IFNβ-1b), glatiramer acetate, mitoxantrone, and natalizumab.

### Cyclophosphamide

Disclosed herein, in certain embodiments, are methods of treating a neurological autoimmune disorder in an individual in need thereof by use of cyclophosphamide. Further disclosed herein, in certain embodiments, is a composition of matter, comprising cyclophosphamide in solution, wherein the cyclophosphamide in solution is reconstituted from lyophilized cyclophosphamide.

Cyclophosphamide (N,N-bis(2-chloroethyl)-1,3,2-oxazaphosphinan-2-amine 2-oxide) is a nitrogen mustard alkylating agent. In certain instances, it is administered to an individual as a prodrug (i.e. an inactive or less active form of a drug that is converted into an active form by metabolism). In certain instances, cyclophosphamide is metabolized in the liver to its active form (4-hydroxycyclophosphamide) and a tautomer of the active form (aldophosphamide). In certain instances, aldophosphamide is converted into (a) carboxyphosphamide (a non-toxic metabolite) by aldehyde dehydrogenase (ALDH); and (b) phosphoramide mustard (a toxic metabolite). In certain instances, ALDH is highly expressed in hematopoietic stem cells. In certain instances, mature or maturing cells (e.g. lymphoid cells, natural killer cells, B-, and T-lymphocytes) express low levels of aldehyde dehydrogenase. In certain instances, phosphoramide mustard is only found in cells with low levels of ALDH. In certain instances, cells with high levels of ALDH predominantly metabolize aldophosphamide into carboxyphosphamide.

In certain instances, phosphoramide mustard catalyzes the formation of crosslinkages in DNA. In certain the crosslinkage is between a dG and another dG at a 5′-d(GAC)-3′. In certain instances, the crosslinkages are between a dG on a first strand of DNA and another dG on the first strand (intrastrand crosslinkages). In certain instances, the crosslinkages are between a dG on a first strand of DNA and a dG on a second strand (interstrand crosslinkages). In certain instances, the formation of both intrastrand crosslinkages and interstrand crosslinkages results in cell death (e.g. apoptosis).

In some embodiments, the cyclophosphamide administered to an individual in need thereof is pulse (or low dose) cyclophosphamide (e.g. 400-1000 mg/m² initially, titrated upwards to reduction in both B and CD4 cells to below 5^{th} percentile for control population).

In some embodiments, the cyclophosphamide administered to an individual in need thereof is high dose cyclophosphamide. In some embodiments, high dose cyclophosphamide is an “upfront” high dose regimen (50 mg/kg IV each day for four consecutive days) of cyclophosphamide, given over a four (4) day period for a total of 200 mg/kg per patient. In certain instances, the high dose cyclophosphamide eliminates most or essentially all maturing and mature elements of an immune system. In certain instances, high dose cyclophosphamide eliminates a non-toxic amount of hematopoietic stem cells. In certain instances, the high dose cyclophosphamide does not eliminate hematopoietic stem cells.

In some embodiments, the cyclophosphamide is formulated as a solution. In some embodiments, the cyclophosphamide solution comprises cyclophosphamide reconstituted from lyophilized cyclophosphamide. In some embodiments, the lyophilized cyclophosphamide is reconstituted in phosphate buffered saline (PBS), a saline solution, water, or combinations thereof. In some embodiments, the concentration of the high dose cyclophosphamide solution is 20 mg/ml. In certain instances, cyclophosphamide is slowly reconstituted at high concentrations (e.g. at concentrations exceeding 15 mg/ml). In certain instances, lyophilized cyclophosphamide is quickly reconstituted at high concentrations (e.g. at concentrations exceeding 15 mg/ml). In some embodiments, the cyclophosphamide is administered to an individual in need thereof intravenously.

### HiGa Treatment

Disclosed herein, in some embodiments, is a method of treating a neurological autoimmune disorder in a individual in need thereof, comprising administering to the individual: (a) about 10 to about 70 mg/kg/day of cyclophosphamide; (b) about 1 to about 10 μg/kg/day of granulocyte colony stimulating factor; and (c) about 10 mg/day to about 80 mg/day of glatiramer acetate; wherein an individual is excluded from treatment if the level of aldehyde dehydrogenase associated with the individual's CD 4+ T cells exceeds a predetermined threshold. In some embodiments, about 50 mg/kg/day of cyclophosphamide is administered to the individual. In some embodiments, about 5 μg/kg/day of granulocyte colony stimulating factor is administered to the individual. In some embodiments, about 40 mg/day of glatiramer acetate is administered to the individual.

In some embodiments, the cyclophosphemide is high dose cyclophosphamide (e.g. 50 mg/kg IV each day for four consecutive days). In some embodiments, the cyclophosphamide is administered each day for about four (4) consecutive days. In certain instances, high dose cyclophosphamide eliminates most or essentially all maturing and mature elements of an immune system. In certain instances, high dose cyclophosphamide eliminates a non-toxic amount of hematopoietic stem cells. In certain instances, high dose cyclophosphamide does not eliminate hematopoietic stem cells.

In some embodiments, the cyclophosphamide is formulated as a solution. In some embodiments, the cyclophosphamide solution comprises cyclophosphamide reconstituted from lyophilized cyclophosphamide. In some embodiments, the lyophilized cyclophosphamide is reconstituted in phosphate buffered saline (PBS), a saline solution, water, or combinations thereof. In some embodiments, the concentration of the cyclophosphamide solution is 20 mg/ml. In some embodiments, the cyclophosphamide is administered to an individual in need thereof intravenously.

In certain instances, an immune system reconstitutes following immunoablation with cyclophosphamide. In certain instances, an immune system reconstitutes with naive, cyclophosphamide-resistant stem cells. In certain instances, individuals treated with cyclophosphamide achieve complete cessation of MS disease activity. In certain instances, the MS in some individuals reactivates and begin to show disease progression. In certain instances, progression of MS following reconstitution is at a slower pace than progression prior to treatment.

In some embodiments, administration of the granulocyte colony stimulating factor (GCSF or CSF 3) is initiated about six (6) days after administration of the cyclophosphamide has been completed. In some embodiments, the granulocyte colony stimulating factor is administered to the individual until the individual's absolute neutrophil count exceeds 1.0×10⁹ cells/L for two (2) consecutive days.

In certain instances, GCSF facilitates recovery of neutrophil counts after the expected transient neutropenia resulting from administration of the cyclophosphemide. GCSF is a colony-stimulating factor hormone. In certain instances, the receptor for GCSF is found on hematopoietic stem cells found in bone marrow. In certain instances, the binding of GCSF to its receptor stimulates the production and release of granulocytes and stem cells by bone marrow. It also stimulates the survival, proliferation, differentiation, and function of neutrophil precursors and mature neutrophils.

In some embodiments, administration of glatiramer acetate is initiated at about thirty (30) days after administration of the cyclophosphamide has been completed. In some embodiments, double dose GA is administered daily. In some embodiments, double dose GA is administered subcutaneously.

Glatiramer acetate (GA) is a synthetic amino acid polymer (4.7-11 kDa) composed of L-alanine, L-lysine, L-glutamic acid, and L-tyrosine, in a molar ratio of 4.2:3.4:1.4:1. It is used in the treatment of RRMS. In certain instances, it takes approximately 3 months of 20 mg GA treatment to reduce the number of lesions and relapses in individuals who have RRMS compared to placebo controls. In certain instances, GA administered at a dosage of 40 mg daily takes less than 3 months to reduce the number of lesions and relapses in individuals who have RRMS compared to controls.

In certain instances, GA binds with high affinity to various class II MHC molecules. In certain instances, the binding of GA to class II MHC molecules causes displacement of antigens that are already bound to the MHC groove, leading to the activation of T suppressor cells. In certain instances, GA induces the production of Th2 regulatory T cells. Further, in certain instances, GA is a general suppressor of autoimmune disease (e.g. it inhibits the onset of experimental uveoretinitis, immune rejection of grafts against host and host against graft disease, and experimental inflammatory bowel disease).

The timing and dose of GA to be used in the methods disclosed herein balances the competing concerns of wanting to avoid any unknown but possible negative effect of GA on a reconstituting immune system following immunoablation with the desire to institute treatment prior to any potential reactivation of neurological autoimmune disorder activity. In certain instances, the median time to a neutrophil count of greater than 500 per ml following immunoablation is approximately 2 weeks. In some embodiments, double dose GA is administered beginning 1 month after immunoablation. In certain instances, administering GA beginning 1 month after immunoablation allows the immune system to reconstitute without any influence by GA; however, it provides sufficient time for GA to vaccinate against recurrence of the neurological autoimmune disorder.

In some embodiments, after 3 months the dose of glatiramer acetate is reduced to about 20 mg/day.

In some embodiments, the glatiramer acetate is administered to the individual indefinitely. In some embodiments, the glatiramer acetate is administered until the individual is no longer at risk of reactivation of the neurological autoimmune disorder. In some embodiments, the glatiramer acetate is administered for about 6 months. In some embodiments, the glatiramer acetate is administered for about 1 year. In some embodiments, the glatiramer acetate is administered for about 2 years. In some embodiments, the glatiramer acetate is administered for about 5 years. In some embodiments, the glatiramer acetate is administered for about 10 years.

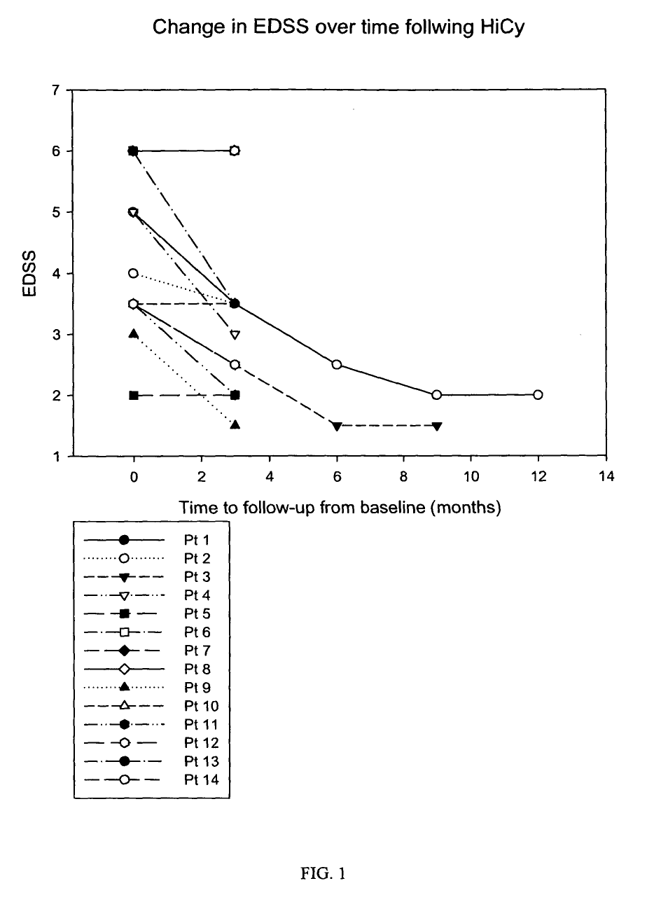

In certain instances, treatment with high dose cyclophosphamide followed by treatment with glatiramer acetate exhibits synergy, that is, MS in individuals treated with only high dose cyclophosphamide reactivates after about three months to about one year, but in combination with treatment with glatiramer acetate, the MS does not reactivate for at least one year, and in certain embodiments, more than one year. See, for example, FIG. 1.

### HiCAT Treatment

Disclosed herein, in some embodiments, is a method of treating multiple sclerosis in a individual in need thereof, comprising administering to the individual: (a) about 10 to about 70 mg/kg/day of cyclophosphamide; (b) about 1 to about 10 μg/kg/day of granulocyte colony stimulating factor; and (c) up to about 5 μg/kg/day of antithymocyte globulin; wherein an individual is excluded from treatment if the individual: does not comply with treatment criteria; is pregnant or will become pregnant; if the level of aldehyde dehydrogenase associated with the individual's CD 4+ T cells exceeds some predetermined threshold for the average CD4+ T cell ALDH activity in the general population; does not, or is unable to, provide informed consent to treatment; or cannot be matched to a supply of packed red blood cells, and platelets. In some embodiments, about 50 mg/kg/day of cyclophosphamide is administered to the individual. In some embodiments, about 5 μg/kg/day of granulocyte colony stimulating factor is administered to the individual. In some embodiments, about 2.5 μg/kg/day of antithymocyte globulin is administered to the individual.

In some embodiments, the cyclophosphemide is high dose cyclophosphamide (e.g. 50 mg/kg IV each day for four consecutive days). In some embodiments, the cyclophosphamide is administered each day for about four (4) consecutive days. In certain instances, high dose cyclophosphamide eliminates most or essentially all maturing and mature elements of an immune system. In certain instances, high dose cyclophosphamide eliminates a non-toxic amount of hematopoietic stem cells. In certain instances, high dose cyclophosphamide does not eliminate hematopoietic stem cells.

In some embodiments, the cyclophosphamide is formulated as a solution. In some embodiments, the cyclophosphamide solution comprises cyclophosphamide reconstituted from lyophilized cyclophosphamide. In some embodiments, the lyophilized cyclophosphamide is reconstituted in phosphate buffered saline (PBS), a saline solution, water, or combinations thereof. In some embodiments, the concentration of the cyclophosphamide solution is 20 mg/ml. In some embodiments, the cyclophosphamide is administered to an individual in need thereof intravenously.

In certain instances, an immune system reconstitutes following immunoablation with cyclophosphamide. In certain instances, an immune system reconstitutes with naive, cyclophosphamide-resistant stem cells. In certain instances, individuals treated with cyclophosphamide achieve complete cessation of MS disease activity.

In certain instances, the MS in some individuals reactivates and begin to show disease progression. In certain instances, MS reactivates following administration of cyclophosphamide partially or fully as a result of a failure of the cyclophosphamide to completely destroy self-reactive T cells. In certain instances, antithymocyte globulins (ATG), deletes intravascular stores of cyclophosphamide resistant T cells. In certain instances, the administration of ATG, either before, overlapping with, concurrently with, or following administration of cyclophosphamide treatment leads to greater immune system tolerance than could be achieved with of cyclophosphamide treatment alone.

In some embodiments, antithymocyte globulin is administered concurrently with the cyclophosphamide. In some embodiments, the antithymocyte globulin is administered before, after, or simultaneously with the cyclophosphamide. In some embodiments, administration of the granulocyte colony stimulating factor is initiated about six (6) days after administration of the cyclophosphamide has been completed.

In some embodiments, administration of the granulocyte colony stimulating factor (GCSF or CSF 3) is initiated about six (6) days after administration of the cyclophosphamide has been completed. In some embodiments, the granulocyte colony stimulating factor is administered to the individual until the individual's absolute neutrophil count exceeds 1.0×10⁹ cells/L for two (2) consecutive days.

In certain instances, GCSF facilitates recovery of neutrophil counts after the expected transient neutropenia resulting from administration of the cyclophosphemide. GCSF is a colony-stimulating factor hormone. In certain instances, the receptor for GCSF is found on hematopoietic stem cells found in bone marrow. In certain instances, the binding of GCSF to its receptor stimulates the production and release of granulocytes and stem cells by bone marrow. It also stimulates the survival, proliferation, differentiation, and function of neutrophil precursors and mature neutrophils.

In some embodiments, the granulocyte colony stimulating factor is administered to the individual until the individual's absolute neutrophil count exceeds 1.0×10⁹ cells/L for two (2) consecutive days.

In some embodiments, the method further comprises administering double dose glatiramer acetate at about thirty (30) days after administration of the cyclophosphamide has been completed. In some embodiments, double dose GA is administered daily. In some embodiments, double dose GA is administered subcutaneously.

In some embodiments, the method further comprises administering (a) other oxazaphosphorines in addition to CPA; (b) various types of antithymocyte antibodies, such as monoclonal and polyclonal antibodies to whole lymphocytes and various T cell specific antigens; and (c) the use of a range of antithymocyte treatments before, concurrent with or following HiCy treatment.

### Individual Control and Drug Distribution Program

In some embodiments, the methods described above further comprise controlling access to the treatment, wherein the controlling comprises a first screen, a second screen, and restricted distribution of the cyclophosphamide.

In some embodiments, the first screen comprises: (a) determining whether the individual complies with treatment criteria; (b) if the individual is female, testing the individual for pregnancy and providing the individual with pregnancy counseling; (c) determining the level of aldehyde dehydrogenase associated with the individual's CD 4+ T cells; and (d) matching the individual with a supply of packed red blood cells, and platelets.

In some embodiments, the treatment criteria comprise inclusion criteria and exclusion criteria. In some embodiments, the inclusion criteria comprise:
a. the individual must be between the ages of 18 and 70 years;
b. the individual must have received a diagnosis of a clinically definite neurological autoimmune disorder (e.g. for MS a definite diagnosis of relapsing-remitting MS according to the McDonald Criteria);
c. for MS, the individual must have two (2) or more total gadolinium enhancing lesions on a brain and/or spinal cord MRI at screening;
d. for MS, the individual must have had at least one clinical relapse in the last year;
e. for MS, for MS, the individual must have an EDSS ranging from 0 to 6 inclusive;
f. the individual must give (and be competent to give) written informed consent prior to any testing under this protocol, including screening tests and evaluations that are not considered part of the individual's routine care; and
g. for females, a negative pregnancy test prior to entry into the study.
   All inclusion criteria must be met in order for an individual to receive treatment.

In some embodiments, the exclusion criteria comprise:
a. any individual at risk of pregnancy;
b. any individual exhibiting cardiac ejection fraction of <45%;
c. any individual exhibiting serum creatinine levels >2.0;
d. any individual who is pre-terminal or moribund;
e. any individual exhibiting bilirubin levels >2.0, and/or transaminases levels >2× normal;
f. any individual with pacemakers and implants who cannot get serial MRIs;
g. any individual with active infections until infection is resolved; or
h. any individual with WBC count <3000 cells/μl; platelets <100,000 cells/μl; and untransfused hemoglobin <10 g/dl.
   If any exclusion criteria are met the individual will be excluded from treatment.

In some embodiments, if the individual is female, the individual must be tested for pregnancy and provided with pregnancy counseling. In some embodiments, pregnancy counseling comprises advising the individual against becoming pregnant. In some embodiments, pregnancy counseling comprises counseling the individual on effective means of birth control (e.g. abstinence; use of condoms, contraceptive sponges, cervical caps, spermicide, hormonal contraception, and intra-uterine devices; hysterectomy; and fallopian tube surgery).

In some embodiments, the first screen comprises determining the level of aldehyde dehydrogenase associated with the individual's CD 4+ T cells. In some embodiments, the first screen comprises determining the level of cell death associated with the individual's PBMCs.

In some embodiments, the first screen further comprises genotyping an individual, and excluding from the cyclophosphamide-based therapies described herein those individuals having polymorphisms in an aldehyde dehydrogenase gene. In certain instances, such polymorphisms in an aldehyde dehydrogenase gene (e.g. ALDH1A1*2, and ALDH1A1*3) partially or fully result in greater than average expression of an aldehyde dehydrogenase gene. In certain instances, greater than average expression of the gene partially or fully results in cells (e.g. T cells) with greater than average levels of an aldehyde dehydrogenase. In certain embodiments, the greater than average levels of an aldehyde dehydrogenase partially or fully results in cells that are resistant to treatment with cyclophosphamide. In certain instances, these individuals will not respond, or will respond poorly, to treatment with cyclophosphamide. In certain instances, African Americans have polymorphisms in an aldehyde dehydrogenase gene (e.g. ALDH1A1*2, and ALDH1A1*3) that partially or fully result in greater than average expression of an aldehyde dehydrogenase gene. In certain instances, African Americans do not respond, or respond poorly, to treatment with cyclophosphamide. See, Scott, et al., Health-Related Effects of Genetic Variations of Alcohol-Metabolizing Enzymes in African Americans, Alcohol Research & Health, Vol. 30, No. 1, at 18 (2007), which is hereby incorporated by reference for such disclosures.

In some embodiments, the first screen comprises matching the individual with a supply of packed red blood cells (RBCs). In certain instances, packed RBCs are preparations of red blood cells that have been separated from blood plasma, leukocytes, or combinations thereof. In some embodiments, the packed RBCs are irradiated. In certain instances, packed RBCs are administered to an individual if the individual suffers from anemia. In certain instances, immunoablation partially or completely results in anemia. In some embodiments, an individual is administered packed RBCs if the individual experiences anemia while undergoing any of the methods described herein.

In some embodiments, the first screen comprises matching the individual with a supply of platelets. In certain instances, platelets are administered to an individual if the individual suffers from thrombocytopenia (or thrombopenia) and thrombocytosis. In certain instances, immunoablation partially or completely results in thrombocytopenia (or thrombopenia) and thrombocytosis. In some embodiments, an individual is administered platelets if the individual experiences thrombocytopenia or thrombocytosis while undergoing any of the methods described herein.

In some embodiments, the second screen comprises continually monitoring the individual for pregnancy, and/or adverse events. An individual experiencing an adverse event is treated appropriately and observed at suitable intervals until the adverse event resolves or stabilizes. Adverse events are reported. The following information regarding each adverse event must be recorded:
a. date and time of onset and resolution (duration);
b. severity (mild, moderate, severe);
   i. Mild—Symptom(s) barely noticeable to the individual or does not make the individual uncomfortable; does not influence performance or functioning; prescription drug not ordinarily needed for relief of symptom(s) but may be given because of personality of the individual.
   ii. Moderate—Symptom(s) of a sufficient severity to make the individual uncomfortable; performance of daily activity is influenced; the individual is able to continue n study; treatment for symptom(s) may be needed.
   iii. Severe—Symptom(s) cause severe discomfort; symptoms cause incapacitation or significant impact on the individual's daily life; severity may cause cessation of treatment with investigational drug; treatment for symptom(s) may be given and/or the individual hospitalized.
c. required treatment or action taken;
d. outcome; and
e. relationship to study drug (not related, unlikely, likely, definite)
   i. Not related—Any reaction that does not follow a reasonable temporal sequence from administration of investigational drug AND that is likely to have been produced by the individual's clinical state or other modes of therapy administered to the individual.
   ii. Unlikely—Any reaction that does not follow a reasonable temporal sequence from administration of investigational drug OR that is likely to have been produced by the individual's clinical state or other modes of therapy administered to the individual.
   iii. Likely—A reaction that follows a reasonable temporal sequence from administration of investigational drug OR that follows a known response pattern to the suspected drug AND that could not be reasonably explained by the known characteristics of the individual's clinical state or other modes of therapy administered to the individual.
   iv. Definite—A reaction that follows a reasonable temporal sequence from administration of investigational drug AND that follows a known response pattern to the suspected drug AND that recurs with rechallenge, and/or is improved by stopping the drug or reducing the dose.

Serious adverse events (SAEs) are classified according to the WHO guidelines as Grade IV and V adverse events. These include signs and symptoms that increase in severity while undergoing treatment with methods disclosed herein. Expected adverse events such as neutropenia and other associated toxicities are carefully monitored and not defined as SAEs unless they are life threatening despite appropriate management.

In some embodiments, an individual is removed from treatment if the individual is pregnant, and/or experiences a sufficiently severe adverse event.

In some embodiments, the restricted distribution of the cyclophosphamide comprises: (a) assigning each individual an identification number; (b) associating an identification number with a container of cyclophosphamide; and (c) administering a container of cyclophosphamide to an individual whose identification number corresponds to the identification number associated with the container. In some embodiments, if an individual passes the first screen and the second screen, the individual is assigned a unique ID. In some embodiments, the unique ID is a numerical ID. In some embodiments, the unique ID is an alphabetic ID. In some embodiments, the unique ID is an alphanumeric ID. In some embodiments, the unique ID is a computer generated ID. In some embodiments, a bar code is generated for the unique ID. In some embodiments, a tamper proof hospital bracelet is affixed with the unique ID and/or bar code. In some embodiments, the hospital bracelet is placed on the individual.

In some embodiments, the unique ID is transmitted to a facility where the cyclophosphamide is prepared for distribution. In some embodiments, preparation for distribution comprises manufacturing the cyclophosphamide, lyophilizing the cyclophosphamide, reconstituting the cyclophosphamide, or combinations thereof. In some embodiments, the unique ID is affixed to a container of cyclophosphamide (e.g. lyophilized, or reconstituted).

In some embodiments, the cyclophosphamide is transmitted to a facility where it will be administered (infusion facility) to the individual whose unique ID matches the unique ID and/or bar code affixed to the container. In some embodiments, if the cyclophosphamide is transmitted to the infusion facility in lyophilized form, the cyclophosphamide is reconstituted at the infusion facility from the lyophilized cyclophosphamide. In some embodiments, the unique ID is affixed to the container (e.g. an IV bag) comprising the cyclophosphamide reconstituted at the infusion facility.

In some embodiments, the cyclophosphamide is administered to the individual whose unique ID matches the unique ID affixed to the container. In some embodiments, the unique ID and/or bar code affixed to the cyclophosphamide are matched to the unique ID and/or bar code on the individual's hospital bracelet.

### Aldehyde Dehydrogenase (ALDH) Assays

Disclosed herein, in certain embodiments, are methods of measuring the level of aldehyde dehydrogenase in a biological sample from the individual. In some embodiments, an individual is selected for treatment with cyclophosphamide if the level of aldehyde dehydrogenase in a plurality of mature and/or maturing cells is below a predetermined threshold. In some embodiments, an individual is selected for treatment with cyclophosphamide if the level of aldehyde dehydrogenase in a plurality of hematopoietic stem cells is exceeds a predetermined threshold.

In some embodiments, an individual is selected for participation in a clinical trial to evaluate the efficacy of cyclophosphamide in treating a neurological autoimmune disorder (e.g. multiple sclerosis, Guillain-Barre syndrome, Lambert-Eaton myasthenic syndrome, myasthenia gravis, transverse myelitis, lupus, or combinations thereof) if the level of aldehyde dehydrogenase in a plurality of mature and/or maturing cells is below a predetermined threshold. In some embodiments, an individual is selected if the level of aldehyde dehydrogenase in a plurality of hematopoietic stem cells is exceeds a predetermined threshold.

In most or essentially all cases, if an individual is not selected for treatment an alternative treatment is selected for the individual. In some embodiments, the alternative treatment is treatment with corticosteroids (e.g. 500 to 1,000 mg of intravenous methylprednisolone followed by a tapering dose of oral prednisone over several weeks), interferons (e.g. IFNβ-1a, and IFNβ-1b), glatiramer acetate, mitoxantrone, natalizumab, alemtuzumab, BG00012 (Biogen), cladribine, dirucotide (MBP8298), fingolimod, laquinimod, rituximab, teriflunomide, ATL1102 (Teva and Antisense Therapeutics), CDP323 (Biogen), daclizumab, estradiol, inosine, neurovax, tovaxin, or combinations thereof.

In some embodiments, the threshold of greater than the 75 percentile for the average CD4+ T cell ALDH activity in the general population will be used to exclude individuals from treatment with high dose cyclophosphamide. In some embodiments such individuals would be treated with high dose cyclophosphamide and relatively higher levels of antithymocyte globulin.

Pre-Treatment Aldehyde Dehydrogenase (ALDH) Assays on Mature and/or Maturing Cells

In some embodiments, the biological sample is mature and/or maturing cells. In some embodiments, the mature and/or maturing cells are white blood cells. In some embodiments, the white blood cells are T cells. In some embodiments, the T cells are CD 4+ T cells.

### Fluorescent Assay

In some embodiments, the level of aldehyde dehydrogenase in a plurality of mature and/or maturing cells is determined by a fluorescent aldehyde dehydrogenase substrate assay. In some embodiments, a plurality of T cells in the plurality of mature and/or maturing cells is activated for 24 to 48 hours using anti-CD3 and anti-CD28 coated magnetic beads. In some embodiments the mature and/or maturing cells are treated with a sublethal dose of cyclophosphamide to induce expression of ALDH. In some embodiments, the plurality of mature and/or maturing cells are stained with a fluorescent aldehyde dehydrogenase substrate (e.g. ALDEFLUOR®), and fluorescent anti-CD4 cell surface markers. In some embodiments, the level of fluorescence is detectable and/or measurable by any suitable manner (e.g. by use of a four color FACS Calibur flow cytometer). In some embodiments, the level of aldehyde dehydrogenase is extrapolated from the level of fluorescence by any suitable manner (e.g. using CellQuest software). In some embodiments, the geometric mean fluorescent intensity (MFI) of the fluorescent aldehyde dehydrogenase substrate is determined for the CD4 cells in the plurality of mature and/or maturing cells.

In some embodiments, the level of aldehyde dehydrogenase is determined more than 12 hours after the plurality of mature and/or maturing cells is collected. In some embodiments, the plurality of mature and/or maturing cells is cryo-preserved. In some embodiments, the plurality of mature and/or maturing cells is thawed in, by way of non-limiting example, Iscove's Modified Dulbecco's Medium (IMDM) with 5% human serum.

In some embodiments, peripheral blood mononuclear cells (PBMCs) are extracted from the plurality of mature and/or maturing cells by any suitable manner (e.g. gradient density centrifugation over Ficoll). In some embodiments, a plurality of T cells in the plurality of PBMCs is activated for 24 to 48 hours using anti-CD3 and anti-CD28 coated magnetic beads. In some embodiments the PBMC are treated with a sublethal dose of cyclophosphamide to induce expression of ALDH. In some embodiments, the plurality of PMBC samples are stained with a fluorescent aldehyde dehydrogenase substrate (e.g. ALDEFLUOR®), and fluorescent anti-CD4 cell surface markers. In some embodiments, the level of fluorescence is detectable and/or measurable by any suitable manner (e.g. by use of a four color FACS Calibur flow cytometer). In some embodiments, the level of aldehyde dehydrogenase is extrapolated from the level of fluorescence by any suitable manner (e.g. using CellQuest software). In some embodiments, the geometric mean fluorescent intensity (MFI) of the fluorescent aldehyde dehydrogenase substrate is determined for the CD4 cells in the plurality of PMBCs.

In some embodiments, the level of aldehyde dehydrogenase is determined more than 12 hours after the plurality of PBMCs is collected. In some embodiments, the plurality of PBMCs is cryo-preserved. In some embodiments, the plurality of PBMCs is thawed in, by way of non-limiting example, Iscove's Modified Dulbecco's Medium (IMDM) with 5% human serum.

### RNA Assay

In some embodiments, the level of aldehyde dehydrogenase is determined by measuring the level of an RNA sequence encoding an aldehyde dehydrogenase. In some embodiments, measuring the level of aldehyde dehydrogenase comprises (a) contacting RNA extracted from the plurality of mature and/or maturing cells with a probe; (b) washing the extracted RNA (e.g. rinsing) with buffer (e.g. FACS buffer) after contact with the probe; and (c) detecting and/or measuring the amount of RNA/probe complex.

In some embodiments, the RNA is extracted from the plurality of mature and/or maturing cells by any suitable manner (e.g. cell lysis followed by phenol-chloroform extraction). In some embodiments, the extracted RNA is hybridized with a probe. In some embodiments, the probe is an oligonucleotide sequence that is homologous to most, essentially all, or part of an RNA sequence encoding aldehyde dehydrogenase. In some embodiments, the probe is isotopically-labeled, radio-labeled, or fluorophore-labeled. In certain instances, the RNA/probe complex is detectable and/or measurable by any suitable manner (e.g. HPLC, fluorescence microscopy, confocal microscopy, microarray scanners, Surface Plasmon Resonance, infrared spectroscopy, or autoradiography). In some embodiments, the probe is purchased from a commercial supplier. In some embodiments, the probe is generated in-house.

In some embodiments, a plurality of peripheral blood mononuclear cells (PBMCs) are extracted from the plurality of mature and/or maturing cells by any suitable manner (e.g. gradient density centrifugation over Ficoll). In some embodiments, a plurality of T cells in the plurality of PBMCs is activated for 24 to 48 hours using anti-CD3 and anti-CD28 coated magnetic beads. In some embodiments the PBMC are treated with a sublethal dose of cyclophosphamide to induce expression of ALDH. In some embodiments, measuring the level of aldehyde dehydrogenase comprises (a) contacting RNA extracted from the plurality of PBMCs with a probe; (b) washing the extracted RNA (e.g. rinsing) with buffer (e.g. FACS buffer) after contact with the probe; and (c) detecting and/or measuring the amount of RNA/probe complex. In some embodiments, the probe is purchased from a commercial supplier. In some embodiments, the probe is generated in-house.

In some embodiments, the RNA is extracted from the plurality of PBMCs by any suitable manner (e.g. cell lysis followed by phenol-chloroform extraction). In some embodiments, the extracted RNA is hybridized with a probe. In some embodiments, the probe is an oligonucleotide sequence that is homologous to most, essentially all, or part of an RNA sequence encoding aldehyde dehydrogenase. In some embodiments, the probe is isotopically-labeled, radio-labeled, or fluorophore-labeled. In certain instances, the RNA/probe complex is detectable and/or measurable by any suitable manner (e.g. HPLC, fluorescence microscopy, confocal microscopy, microarray scanners, Surface Plasmon Resonance, infrared spectroscopy, or autoradiography). In some embodiments, the probe is purchased from a commercial supplier. In some embodiments, the probe is generated in-house.

### Antibody Assay

In some embodiments, the level of aldehyde dehydrogenase is measured by contacting a plurality of mature and/or maturing cells with antibodies to aldehyde dehydrogenase. In some embodiments, measuring the level of aldehyde dehydrogenase comprises (a) lysing a plurality of mature and/or maturing cells; (b) contacting the lysate from a plurality of mature and/or maturing cells with antibodies to aldehyde dehydrogenase; (c) washing the antibody-lysate mixture (e.g. rinsing) with buffer (e.g. FACS buffer) after contact with the antibodies; and (d) detecting and/or measuring the amount of antibody/aldehyde dehydrogenase complex. In some embodiments, the antibodies are purchased from a commercial supplier. In some embodiments, the antibodies are generated in-house. For methods of generating antibodies, see Kohler et al., Nature, 256:495 (1975); U.S. Pat. No. 4,816,567; or Goding, Monoclonal Antibodies: Principles and Practice (Academic Press, 1986); Ward et al., Nature 341: 544-546 (1989); Huse et al., Science 246: 1275-1281 (1989); McCafferty et al., Nature 348: 552-554 (1990); Clackson et al., Nature, 352:624-628 (1991) Marks et al., J. Mol. Biol., 222:581-597 (1991) all of which are hereby incorporated by reference for such disclosure. In some embodiments, the lysate is incubated on ice during the contact with the antibodies. In some embodiments, the antibody is isotopically-labeled, radio-labeled, fluorophore-labeled, or biotinylated. In some embodiments, the fluorophore is fluorescein. In certain instances, the cell surface marker/antibody complex is detectable and/or measurable by any suitable manner (e.g. HPLC, fluorescence microscopy, confocal microscopy, microarray scanners, Surface Plasmon Resonance, infrared spectroscopy, or autoradiography).

In some embodiments, the level of aldehyde dehydrogenase is measured by contacting the plurality of PBMCs with antibodies to aldehyde dehydrogenase. In some embodiments, measuring the level of aldehyde dehydrogenase comprises (a) lysing a plurality of PBMCs; (b) contacting the lysate with antibodies to aldehyde dehydrogenase; (c) washing the antibody-lysate mixture (e.g. rinsing) with buffer (e.g. FACS buffer) after contact with the antibodies; and (d) detecting and/or measuring the amount of antibody/aldehyde dehydrogenase complex. In some embodiments, the antibodies are purchased from a commercial supplier. In some embodiments, the antibodies are generated in-house. For methods of generating antibodies, see Kohler et al., Nature, 256:495 (1975); U.S. Pat. No. 4,816,567; or Goding, Monoclonal Antibodies: Principles and Practice (Academic Press, 1986); Ward et al., Nature 341: 544-546 (1989); Huse et al., Science 246: 1275-1281 (1989); McCafferty et al., Nature 348: 552-554 (1990); Clackson et al., Nature, 352:624-628 (1991) Marks et al., J. Mol. Biol., 222:581-597 (1991) all of which are hereby incorporated by reference for such disclosure. In some embodiments, the plurality of PBMCs is incubated on ice during the contact with the antibodies. In some embodiments, the antibody is isotopically-labeled, radio-labeled, fluorophore-labeled, or biotinylated. In some embodiments, the fluorophore is fluorescein. In certain instances, the cell surface marker/antibody complex is detectable and/or measurable by any suitable manner (e.g. HPLC, fluorescence microscopy, confocal microscopy, microarray scanners, Surface Plasmon Resonance, infrared spectroscopy, or autoradiography).

Monitoring of Individuals Undergoing Cyclophosphamide Treatment with Aldehyde Dehydrogenase (ALDH) Assays on Mature and/or Maturing Cells

Further disclosed herein, in certain embodiments, is a method of monitoring an individual being administered cyclophosphamide, comprising determining the level of aldehyde dehydrogenase in at least a first plurality of mature and/or maturing cells and a second plurality of mature and/or maturing cells, wherein the first plurality of mature and/or maturing cells and the second plurality of mature and/or maturing cells are taken from the individual at different times (e.g. sample 1 is taken before the administration of cyclophosphamide, and sample 2 is taken 96 hours after the administration of cyclophosphamide is completed).

In some embodiments, the method further comprises discontinuing treatment if the level of aldehyde dehydrogenase observed in mature and/or maturing cells exceeds a predetermined threshold. In some embodiments, the method further comprises selecting an alternative treatment if the level of aldehyde dehydrogenase observed in mature and/or maturing cells exceeds a predetermined threshold. In some embodiments, the method further comprises altering treatment based on the level of aldehyde dehydrogenase observed in a biological sample. In some embodiments, if the level of ALDH increases, the dose of cyclophosphamide is increased. In some embodiments, if the level of ALDH decreases, the dose of cyclophosphamide is decreased.

Further disclosed herein, in certain embodiments, is a method of monitoring an individual being administered cyclophosphamide, comprising determining the level of aldehyde dehydrogenase in at least a first plurality of PBMCs and a second plurality of PBMCs, wherein the first plurality of PBMCs and the second plurality of PBMCs are taken from the individual at different times (e.g. sample 1 is taken before the administration of cyclophosphamide, and sample 2 is taken 96 hours after the administration of cyclophosphamide is completed).

In some embodiments, the method further comprises discontinuing treatment if the level of aldehyde dehydrogenase observed in a plurality of PBMCs exceeds a predetermined threshold. In some embodiments, the method further comprises selecting an alternative treatment if the level of aldehyde dehydrogenase observed in a plurality of PBMCs exceeds a predetermined threshold. In some embodiments, the method further comprises altering treatment based on the level of aldehyde dehydrogenase observed in a plurality of PBMCs. In some embodiments, if the level of ALDH increases, the dose of cyclophosphamide is increased. In some embodiments, if the level of ALDH decreases, the dose of cyclophosphamide is decreased.

### Pre-Treatment Aldehyde Dehydrogenase (ALDH) Assays on Bone Marrow

In some embodiments, the biological sample is bone marrow (e.g. red bone marrow, yellow bone marrow, hematopoietic stem cells, or combinations thereof). In certain instances, the bone marrow is obtained by any suitable manner (e.g. bone marrow biopsy, bone marrow aspiration.

### Fluorescent Assays

In some embodiments, the level of aldehyde dehydrogenase in bone marrow is determined by a fluorescent aldehyde dehydrogenase substrate assay. In some embodiments the bone marrow is treated with a sublethal dose of cyclophosphamide to induce expression of ALDH. In some embodiments, the bone marrow is stained with a fluorescent aldehyde dehydrogenase substrate (e.g. ALDEFLUOR). In some embodiments, the level of fluorescence is detectable and/or measurable by any suitable manner (e.g. by use of a four color FACS Calibur flow cytometer). In some embodiments, the level of aldehyde dehydrogenase is extrapolated from the level of fluorescence by any suitable manner (e.g. using CellQuest software).

In some embodiments, a plurality of hematopoietic stem cells is separated from the rest of the bone marrow by any suitable manner. In some embodiments, the plurality of hematopoietic stem cells is treated with a sublethal dose of cyclophosphamide to induce expression of ALDH. In some embodiments, a plurality of hematopoietic stem cells is stained with a fluorescent aldehyde dehydrogenase substrate (e.g. ALDEFLUOR). In some embodiments, the level of fluorescence is detectable and/or measurable by any suitable manner (e.g. by use of a four color FACS Calibur flow cytometer). In some embodiments, the level of aldehyde dehydrogenase is extrapolated from the level of fluorescence by any suitable manner (e.g. using CellQuest software).

### RNA Assays

In some embodiments, the level of aldehyde dehydrogenase is determined by measuring the level of an RNA sequence encoding an aldehyde dehydrogenase. In some embodiments, measuring the level of aldehyde dehydrogenase comprises (a) contacting RNA extracted from the bone marrow with a probe; (b) washing the RNA (e.g. rinsing) with buffer (e.g. FACS buffer) after contact with the probe; and (c) detecting and/or measuring the amount of RNA/probe complex. In some embodiments, the probe is purchased from a commercial supplier. In some embodiments, the probe is generated in-house.

In some embodiments, the RNA is extracted from bone marrow by any suitable manner (e.g. cell lysis followed by phenol-chloroform extraction). In some embodiments, the extracted RNA is hybridized with a probe. In some embodiments, the probe is an oligonucleotide sequence that is homologous to most, essentially all, or part of an RNA sequence encoding aldehyde dehydrogenase. In some embodiments, the probe is isotopically-labeled, radio-labeled, or fluorophore-labeled. In certain instances, the RNA/probe complex is detectable and/or measurable by any suitable manner (e.g. HPLC, fluorescence microscopy, confocal microscopy, microarray scanners, Surface Plasmon Resonance, infrared spectroscopy, or autoradiography).

In some embodiments, a plurality of hematopoietic stem cells are separated from the rest of the bone marrow by any suitable manner. In some embodiments, measuring the level of aldehyde dehydrogenase comprises (a) contacting RNA extracted from a plurality of hematopoietic stem cells with a probe; (b) washing the RNA (e.g. rinsing) with buffer (e.g. FACS buffer) after contact with the probe; and (c) detecting and/or measuring the amount of RNA/probe complex. In some embodiments, the probe is purchased from a commercial supplier. In some embodiments, the probe is generated in-house.

In some embodiments, hematopoietic stem cells are separated from the rest of the bone marrow by any suitable manner. In some embodiments, the RNA is extracted from the hematopoietic stem cells by any suitable manner (e.g. cell lysis followed by phenol-chloroform extraction). In some embodiments, the extracted RNA is hybridized with a probe. In some embodiments, the probe is an oligonucleotide sequence that is homologous to most, essentially all, or part of an RNA sequence encoding aldehyde dehydrogenase. In some embodiments, the probe is isotopically-labeled, radio-labeled, or fluorophore-labeled. In certain instances, the RNA/probe complex is detectable and/or measurable by any suitable manner (e.g. HPLC, fluorescence microscopy, confocal microscopy, microarray scanners, Surface Plasmon Resonance, infrared spectroscopy, or autoradiography).

### Antibody Assays

In some embodiments, the level of aldehyde dehydrogenase is measured by contacting the bone marrow with antibodies to aldehyde dehydrogenase. In some embodiments, measuring the level of aldehyde dehydrogenase comprises (a) lysing the bone marrow sample; (b) contacting the lysate with antibodies to aldehyde dehydrogenase; (c) washing antibody-lysate mixture (e.g. rinsing) with buffer (e.g. FACS buffer) after contact with the antibodies; and (d) detecting and/or measuring the amount of antibody/aldehyde dehydrogenase complex. In some embodiments, the antibodies are purchased from a commercial supplier. In some embodiments, the antibodies are generated in-house. For methods of generating antibodies, see Kohler et al., Nature, 256:495 (1975); U.S. Pat. No. 4,816,567; or Goding, Monoclonal Antibodies: Principles and Practice (Academic Press, 1986); Ward et al., Nature 341: 544-546 (1989); Huse et al., Science 246: 1275-1281 (1989); McCafferty et al., Nature 348: 552-554 (1990); Clackson et al., Nature, 352:624-628 (1991) Marks et al., J. Mol. Biol., 222:581-597 (1991) all of which are hereby incorporated by reference for such disclosure. In some embodiments, the bone marrow is incubated on ice during the contact with the antibodies. In some embodiments, the antibody is isotopically-labeled, radio-labeled, fluorophore-labeled, or biotinylated. In some embodiments, the fluorophore is fluorescein. In certain instances, the aldehyde dehydrogenase/antibody complex is detectable and/or measurable by any suitable manner (e.g. HPLC, fluorescence microscopy, confocal microscopy, microarray scanners, Surface Plasmon Resonance, infrared spectroscopy, or autoradiography).

In some embodiments, the level of aldehyde dehydrogenase is measured by contacting a plurality of hematopoietic stem cells with antibodies to aldehyde dehydrogenase. In some embodiments, measuring the level of aldehyde dehydrogenase comprises (a) lysing the plurality of hematopoietic stem cells; (b) contacting the lysate with antibodies to aldehyde dehydrogenase; (c) washing antibody-lysate mixture (e.g. rinsing) with buffer (e.g. FACS buffer) after contact with the antibodies; and (d) detecting and/or measuring the amount of antibody/aldehyde dehydrogenase complex. In some embodiments, the antibodies are purchased from a commercial supplier. In some embodiments, the antibodies are generated in-house. For methods of generating antibodies, see Kohler et al., Nature, 256:495 (1975); U.S. Pat. No. 4,816,567; or Goding, Monoclonal Antibodies: Principles and Practice (Academic Press, 1986); Ward et al., Nature 341: 544-546 (1989); Huse et al., Science 246: 1275-1281 (1989); McCafferty et al., Nature 348: 552-554 (1990); Clackson et al., Nature, 352:624-628 (1991) Marks et al., J. Mol. Biol., 222:581-597 (1991) all of which are hereby incorporated by reference for such disclosure. In some embodiments, the lysate is incubated on ice during the contact with the antibodies. In some embodiments, the antibody is isotopically-labeled, radio-labeled, fluorophore-labeled, or biotinylated. In some embodiments, the fluorophore is fluorescein. In certain instances, the aldehyde dehydrogenase/antibody complex is detectable and/or measurable by any suitable manner (e.g. HPLC, fluorescence microscopy, confocal microscopy, microarray scanners, Surface Plasmon Resonance, infrared spectroscopy, or autoradiography).

Monitoring of Individuals Undergoing Cyclophosphamide Treatment with Aldehyde Dehydrogenase (ALDH) Assays on Bone Marrow

Further disclosed herein, in certain embodiments, is a method of monitoring an individual being administered cyclophosphamide, comprising determining the level of aldehyde dehydrogenase in at least a first bone marrow sample and a second bone marrow sample, wherein the first bone marrow sample and the second bone marrow sample are taken from the individual at different times (e.g. sample 1 is taken before the administration of cyclophosphamide, and sample 2 is taken 96 hours after the administration of cyclophosphamide is completed).

In some embodiments, the method further comprises discontinuing treatment if the level of aldehyde dehydrogenase observed in a bone marrow sample is below a predetermined threshold. In some embodiments, the method further comprises selecting an alternative treatment if the level of aldehyde dehydrogenase observed in a bone marrow sample is below a predetermined threshold. In some embodiments, the method further comprises altering treatment based on the level of aldehyde dehydrogenase observed in bone marrow sample. In some embodiments, if the level of ALDH increases, the dose of cyclophosphamide is increased. In some embodiments, if the level of ALDH decreases, the dose of cyclophosphamide is decreased.

Further disclosed herein, in certain embodiments, is a method of monitoring an individual being administered cyclophosphamide, comprising determining the level of aldehyde dehydrogenase in at least a first plurality of hematopoietic stem cells and a second hematopoietic stem cells, wherein the first plurality of hematopoietic stem cells and the second hematopoietic stem cells are taken from the individual at different times (e.g. sample 1 is taken before the administration of cyclophosphamide, and sample 2 is taken 96 hours after the administration of cyclophosphamide is completed).

In some embodiments, the method further comprises discontinuing treatment if the level of aldehyde dehydrogenase observed in a plurality of hematopoietic stem cells is below a predetermined threshold. In some embodiments, the method further comprises selecting an alternative treatment if the level of aldehyde dehydrogenase observed in a plurality of hematopoietic stem cells is below a predetermined threshold. In some embodiments, the method further comprises altering treatment based on the level of aldehyde dehydrogenase observed in a plurality of hematopoietic stem cells. In some embodiments, if the level of ALDH increases, the dose of cyclophosphamide is increased. In some embodiments, if the level of ALDH decreases, the dose of cyclophosphamide is decreased.

### Cyclophosphamide-Induced Cell Death Assays

Pre-Treatment Cell Death Assays on Mature and/or Maturing Cells

Disclosed herein, in certain embodiments, are methods of measuring the level of cyclophosphamide-induced cell death (e.g. apoptosis or necrosis) in a plurality of mature and/or maturing cells from an individual. In some embodiments, an individual is selected for treatment with cyclophosphamide if the level of cell death in a plurality of mature and/or maturing cells from the individual exceeds a predetermined threshold. In some embodiments, an individual is selected for participation in a clinical trial to evaluate the efficacy of cyclophosphamide in treating a neurological autoimmune disorder (e.g. multiple sclerosis, Guillain-Barre syndrome, Lambert-Eaton myasthenic syndrome, myasthenia gravis, transverse myelitis, lupus, or combinations thereof) if the level of cyclophosphamide-induced cell death in a plurality of mature and/or maturing cells from the individual exceeds a predetermined threshold.

In some embodiments, a plurality of mature and/or maturing cells is collected from the individual by any suitable manner. In some embodiments, the plurality of mature and/or maturing cells is contacted with cyclophosphamide. In some embodiments, the plurality of mature and/or maturing cells is contacted with the cyclophosphamide for about 24 hours. In some embodiments, the level of cell death is compared to that of a control. In some embodiments, the control is a plurality of mature and/or maturing cells that exhibits a known level of cell death following contact with cyclophosphamide. In some embodiments, the control is the average level of cell death seen in a plurality of mature and/or maturing cells following contact with cyclophosphamide. In some embodiments, if the level of cell death in a biological sample from the individual is less than the control, an alternative treatment is selected for the individual.

Disclosed herein, in certain embodiments, are methods of measuring the level of cyclophosphamide-induced cell death (e.g. apoptosis or necrosis) in a plurality of PBMCs from an individual. In some embodiments, an individual is selected for treatment with cyclophosphamide if the level of cell death in a plurality of PBMCs from the individual exceeds a predetermined threshold. In some embodiments, an individual is selected for participation in a clinical trial to evaluate the efficacy of cyclophosphamide in treating a neurological autoimmune disorder (e.g. multiple sclerosis, Guillain-Barre syndrome, Lambert-Eaton myasthenic syndrome, myasthenia gravis, transverse myelitis, lupus, or combinations thereof) if the level of cyclophosphamide-induced cell death in a plurality of PBMCs from the individual exceeds a predetermined threshold.

In some embodiments, Peripheral Blood Mononuclear Cell (PBMCs) are separated from a plurality of mature and/or maturing cells by any suitable manner (e.g. gradient density centrifugation over Ficoll). In some embodiments, the PBMCs are contacted with cyclophosphamide. In some embodiments, the PBMCs are contacted with the cyclophosphamide for about 24 hours. In some embodiments, the level of cell death is compared to that of a control. In some embodiments, the control is a plurality of PBMCs that exhibits a known level of cell death following contact with cyclophosphamide. In some embodiments, the control is the average level of cell, death seen in plurality of PBMCs following contact with cyclophosphamide. In some embodiments, if the level of cell death in a plurality of PBMCs from the individual is less than the control, an alternative treatment is selected for the individual.

Monitoring of Individuals Undergoing Cyclophosphamide Treatment with Cell Death Assays on Mature and/or Maturing Cells

Further disclosed herein, in certain embodiments, is a method of monitoring an individual being administered cyclophosphamide, comprising determining the level of cell death in at least a first plurality of mature and/or maturing cells sample and a second plurality of mature and/or maturing cells, wherein the first plurality of mature and/or maturing cells and the second plurality of mature and/or maturing cells are taken from the individual at different times (e.g. sample 1 is taken before the administration of cyclophosphamide, and sample 2 is taken 96 hours after the administration of cyclophosphamide is completed).

In some embodiments, the method further comprises discontinuing treatment if the level of cell death observed in a plurality of mature and/or maturing cells is below a predetermined threshold. In some embodiments, the method further comprises selecting an alternative treatment if the level of cell death observed in a plurality of mature and/or maturing cells is below of the predetermined threshold. In some embodiments, the method further comprises altering treatment based on the level of cell death observed in a plurality of mature and/or maturing cells. In some embodiments, if the level of cell death increases, the dose of cyclophosphamide is decreased. In some embodiments, if the level of ALDH decreases, the dose of cyclophosphamide is increased.

Further disclosed herein, in certain embodiments, is a method of monitoring an individual being administered cyclophosphamide, comprising determining the level of cell death in at least a first plurality of PBMCs and a second plurality of PBMCs, wherein the first plurality of PBMCs and the second plurality of PBMCs are taken from the individual at different times (e.g. sample 1 is taken before the administration of cyclophosphamide, and sample 2 is taken 96 hours after the administration of cyclophosphamide is completed).

In some embodiments, the method further comprises discontinuing treatment if the level of cell death observed in plurality of PBMCs is below a predetermined threshold. In some embodiments, the method further comprises selecting an alternative treatment if the level of cell death observed in a plurality of PBMCs is below of the predetermined threshold. In some embodiments, the control is a plurality of PBMCs that exhibits a known level of cell death following contact with cyclophosphamide. In some embodiments, the method further comprises altering treatment based on the level of cell death observed in a plurality of PBMCs. In some embodiments, if the level of cell death increases, the dose of cyclophosphamide is decreased. In some embodiments, if the level of ALDH decreases, the dose of cyclophosphamide is increased.

### Pre-Treatment Cell Death Assays on Bone Marrow

Disclosed herein, in certain embodiments, are methods of measuring the level of cyclophosphamide-induced cell death (e.g. apoptosis) in bone marrow from an individual. In some embodiments, an individual is selected for treatment with cyclophosphamide if the level of cell death in the bone marrow from the individual is below a predetermined threshold. In some embodiments, an individual is selected for participation in a clinical trial to evaluate the efficacy of cyclophosphamide in treating a neurological autoimmune disorder (e.g. multiple sclerosis, Guillain-Barre syndrome, Lambert-Eaton myasthenic syndrome, myasthenia gravis, transverse myelitis, lupus, or combinations thereof) if the level of cyclophosphamide-induced cell death in the bone marrow from the individual is below a predetermined threshold.

In some embodiments, bone marrow is contacted with cyclophosphamide. In some embodiments, the bone marrow is contacted with the cyclophosphamide for about 24 hours. In some embodiments, the control is bone marrow that exhibits a known level of cell death following contact with cyclophosphamide. In some embodiments, the control is the average level of cell death seen in bone marrow following contact with cyclophosphamide. In some embodiments, if the level of cell death in bone marrow from the individual is greater than the control, an alternative treatment is selected for the individual.

In some embodiments, a plurality of hematopoietic stem cells is separated from the bone marrow by any suitable manner. In some embodiments, the plurality of hematopoietic stem cells is contacted with cyclophosphamide. In some embodiments, the plurality of hematopoietic stem cells is contacted with the cyclophosphamide for about 24 hours. In some embodiments, the level of cell death is compared to that of a control. In some embodiments, the control is a plurality of hematopoietic stem cells that exhibits a known level of cell death following contact with cyclophosphamide. In some embodiments, the control is the average level of cell death seen in a hematopoietic stem cells following contact with cyclophosphamide. In some embodiments, if the level of cell death in a plurality of hematopoietic stem cells from the individual is greater than the control, an alternative treatment is selected for the individual.

Monitoring of Individuals Undergoing Cyclophosphamide Treatment with Cell Death Assays on Bone Marrow

Further disclosed herein, in certain embodiments, is a method of monitoring an individual being administered cyclophosphamide, comprising determining the level of cell death in at least a first bone marrow sample and a second bone marrow sample, wherein the first bone marrow sample and the second bone marrow sample are taken from the individual at different times (e.g. sample 1 is taken before the administration of cyclophosphamide, and sample 2 is taken 96 hours after the administration of cyclophosphamide is completed).

In some embodiments, first bone marrow sample and the second bone marrow sample are contacted with cyclophosphamide (e.g. for about 24 hours). In some embodiments, the method further comprises discontinuing treatment if the level of cell death observed in a bone marrow sample exceeds a predetermined threshold. In some embodiments, the method further comprises selecting an alternative treatment if the level of cell death observed in a bone marrow sample exceeds the predetermined threshold.

In some embodiments, the method further comprises altering treatment based on the level of cell death observed in a bone marrow sample. In some embodiments, if the level of cell death increases, the dose of cyclophosphamide is decreased. In some embodiments, if the level of ALDH decreases, the dose of cyclophosphamide is increased.

In some embodiments, a plurality of hematopoietic stem cells is separated from the first bone marrow sample and the second bone marrow sample by any suitable manner. In some embodiments, the plurality of hematopoietic stem cells from the first bone marrow sample and the plurality of hematopoietic stem cells from the second bone marrow sample is contacted with cyclophosphamide (e.g. for about 24 hours). In some embodiments, the method further comprises discontinuing treatment if the level of cell death observed in either the first plurality or the second plurality exceeds a predetermined threshold. In some embodiments, the method further comprises selecting an alternative treatment if the level of cell death observed in either the first plurality or the second plurality exceeds the predetermined threshold. In some embodiments, the method further comprises altering treatment based on the level of cell death observed in a second plurality of hematopoietic stem cells. In some embodiments, if the level of cell death increases, the dose of cyclophosphamide is decreased. In some embodiments, if the level of ALDH decreases, the dose of cyclophosphamide is increased.

### Formulations of Pharmaceutical Compositions

In some embodiments, pharmaceutical compositions are formulated in a conventional manner using one or more physiologically acceptable carriers including, e.g., excipients and auxiliaries which facilitate processing of the active compounds into preparations which are suitable for pharmaceutical use. In certain embodiments, proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

A pharmaceutical composition, as used herein, refers to a mixture of a compound described herein (e.g. cyclophosphamide, glatiramer acetate, granulocyte colony stimulating factor, and antithymocyte globulin), with other chemical components, such as carriers, stabilizers, diluents, dispersing agents, suspending agents, thickening agents, and/or excipients. In certain instances, the pharmaceutical composition facilitates administration of the compound to an individual or cell. In certain embodiments of practicing the methods of treatment or use provided herein, therapeutically effective amounts of compounds described herein are administered in a pharmaceutical composition to an individual having a disease, disorder, or condition to be treated. In specific embodiments, the individual is a human. As discussed herein, the therapeutic compounds described herein are either utilized singly or in combination with one or more additional therapeutic agents.

In some embodiments, the pharmaceutical formulations described herein are administered to an individual in any manner, including one or more of multiple administration routes, such as, by way of non-limiting example, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

Pharmaceutical compositions of a compound described herein (e.g. cyclophosphamide, glatiramer acetate, granulocyte colony stimulating factor, and antithymocyte globulin) are optionally manufactured in a conventional manner, such as, by way of example only, by means of conventional mixing, dissolving, reconstituting, granulating, dragee-making, levigating, emulsifying, encapsulating, entrapping or compression processes.

In some embodiments, a pharmaceutical compositions described herein includes one or more agents described herein described (e.g. cyclophosphamide, glatiramer acetate, granulocyte colony stimulating factor, and antithymocyte globulin), as an active ingredient in free-acid or free-base form, or in a pharmaceutically acceptable salt form. In some embodiments, the compounds described herein are utilized as an N-oxide or in a crystalline or amorphous form (i.e., a polymorph). In certain embodiments, an active metabolite or prodrug of a compound described herein is utilized. In some situations, a compound described herein exists as tautomers. All tautomers are included within the scope of the compounds presented herein. In certain embodiments, a compound described herein exists in an unsolvated or solvated form, wherein solvated forms comprise any pharmaceutically acceptable solvent, e.g., water, ethanol, and the like. The solvated forms of the compounds presented herein are also considered to be disclosed herein.

A “carrier” includes, in some embodiments, a pharmaceutically acceptable excipient and is selected on the basis of compatibility with compounds disclosed herein, such as, compounds of any of Formulas I-V, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Moreover, in some embodiments, the pharmaceutical compositions described herein are formulated as a dosage form. As such, in some embodiments, provided herein is a dosage form comprising a compound herein described (e.g. cyclophosphamide, glatiramer acetate, granulocyte colony stimulating factor, and antithymocyte globulin) suitable for administration to an individual. In certain embodiments, suitable dosage forms include, by way of non-limiting example, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

The pharmaceutical solid dosage forms described herein optionally include an additional therapeutic compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In some aspects, using standard coating procedures, such as those described in Remington's Pharmaceutical Sciences, 20th Edition (2000), a film coating is provided around the formulation of the compound of any of Formula I-V. In one embodiment, a compound described herein is in the form of a particle and some or all of the particles of the compound are coated. In certain embodiments, some or all of the particles of a compound described herein are microencapsulated. In some embodiment, the particles of the compound described herein are not microencapsulated and are uncoated.

In some embodiments, the pharmaceutical composition described herein is in unit dosage forms suitable for single administration of precise dosages. In unit dosage form, the formulation is divided into unit doses containing appropriate quantities of one or more compound. In some embodiments, the unit dosage is in the form of a package containing discrete quantities of the formulation. Non-limiting examples are packaged tablets or capsules, and powders in vials or ampoules. Aqueous suspension compositions are optionally packaged in single-dose non-reclosable containers. In some embodiments, multiple-dose re-closeable containers are used. In certain instances, multiple dose containers comprise a preservative in the composition. By way of example only, formulations for parenteral injection are presented in unit dosage form, which include, but are not limited to ampoules, or in multi-dose containers, with an added preservative.

### Combinations

In some embodiments, it is appropriate to administer at least one therapeutic agent described herein in combination with another therapeutic agent. Or, by way of example only, the benefit experienced by an individual is increased by administering one of the compounds described herein with another therapeutic agent (which also includes a therapeutic regimen) that also has therapeutic benefit. In any case, regardless of the disease, disorder or condition being treated, the overall benefit experienced by the individual is in some embodiments additive of the two therapeutic agents or in other embodiments, the individual experiences a synergistic benefit.

In some embodiments, the particular choice of compounds depends upon the diagnosis of the attending physicians and their judgment of the condition of the individual and the appropriate treatment protocol. The compounds are optionally administered concurrently (e.g., simultaneously, essentially simultaneously or within the same treatment protocol) or sequentially, depending upon the nature of the disease, disorder, or condition, the condition of the individual, and the actual choice of compounds used. In certain instances, the determination of the order of administration, and the number of repetitions of administration of each therapeutic agent during a treatment protocol, is based on an evaluation of the disease being treated and the condition of the individual.

In some embodiments, therapeutically-effective dosages vary when the drugs are used in treatment combinations. Methods for experimentally determining therapeutically-effective dosages of drugs and other agents for use in combination treatment regimens are described in the literature. For example, the use of metronomic dosing, i.e., providing more frequent, lower doses in order to minimize toxic side effects, has been described extensively in the literature. Combination treatment further includes periodic treatments that start and stop at various times to assist with the clinical management of the individual.

In some embodiments of the combination therapies described herein, dosages of the co-administered compounds vary depending on the type of co-drug employed, on the specific drug employed, on the disease or condition being treated and so forth. In addition, when co-administered with one or more biologically active agents, the compound provided herein is optionally administered either simultaneously with the biologically active agent(s), or sequentially. In certain instances, if administered sequentially, the attending physician will decide on the appropriate sequence of therapeutic compound described herein in combination with the additional therapeutic agent.

The multiple therapeutic agents (at least one of which is a therapeutic compound described herein) are optionally administered in any order or even simultaneously. If simultaneously, the multiple therapeutic agents are optionally provided in a single, unified form, or in multiple forms (by way of example only, either as a single pill or as two separate pills). In certain instances, one of the therapeutic agents is optionally given in multiple doses. In other instances, both are optionally given as multiple doses. If not simultaneous, the timing between the multiple doses is any suitable timing, e.g., from more than zero weeks to less than four weeks. In some embodiments, the additional therapeutic agent is utilized to achieve remission (partial or complete) of a neurological autoimmune disorder, whereupon the therapeutic agent described herein (e.g., cyclophosphamide, glatiramer acetate, granulocyte colony stimulating factor, and antithymocyte globulin) is subsequently administered. In addition, the combination methods, compositions and formulations are not to be limited to the use of only two agents; the use of multiple therapeutic combinations are also envisioned (including two or more therapeutic compounds described herein).

In some embodiments, a dosage regimen to treat, prevent, or ameliorate the condition(s) for which relief is sought, is modified in accordance with a variety of factors. These factors include the disorder from which the individual suffers, as well as the age, weight, sex, diet, and medical condition of the individual. Thus, in various embodiments, the dosage regimen actually employed varies and deviates from the dosage regimens set forth herein.

In some embodiments, the pharmaceutical agents which make up the combination therapy disclosed herein are provided in a combined dosage form or in separate dosage forms for substantially simultaneous administration. In certain embodiments, the pharmaceutical agents that make up the combination therapy are administered sequentially, with either therapeutic compound being administered by a regimen calling for two-step administration. In some embodiments, two-step administration regimen calls for sequential administration of the active agents or spaced-apart administration of the separate active agents. In certain embodiments, the time period between the multiple administration steps varies, by way of non-limiting example, from a few minutes to several hours, depending upon the properties of each pharmaceutical agent, such as potency, solubility, bioavailability, plasma half-life and kinetic profile of the pharmaceutical agent.

In some embodiments, the compounds described herein and combination therapies are administered before, during or after the occurrence of a disease or condition. Timing of administering the composition containing a compound is optionally varied to suit the needs of the individual treated. Thus, in certain embodiments, the compounds are used as a prophylactic and are administered continuously to individuals with a propensity to develop conditions or diseases in order to prevent the occurrence of the disease or condition. In some embodiments, the compounds and compositions are administered to a individual during or as soon as possible after the onset of the symptoms. The administration of the compounds is optionally initiated within the first 48 hours of the onset of the symptoms, within the first 6 hours of the onset of the symptoms, or within 3 hours of the onset of the symptoms. The initial administration is achieved by any route practical, such as, for example, an intravenous injection, a bolus injection, infusion over 5 minutes to about 5 hours, a pill, a capsule, transdermal patch, buccal delivery, and the like, or combination thereof. In some embodiments, the compound should be administered as soon as is practicable after the onset of a disease or condition is detected or suspected, and for a length of time necessary for the treatment of the disease, such as, for example, from more than 1 month to about 3 months. The length of treatment is optionally varied for each individual based on known criteria. In exemplary embodiments, the compound or a formulation containing the compound is administered for at least 2 weeks, between more than 1 month to about 5 years, or from more than 1 month to about 3 years.

In some embodiments, therapeutic agents are combined with or utilized in combination with one or more of the following therapeutic agents in any combination: corticosteroids (e.g. 500 to 1,000 mg of intravenous methylprednisolone followed by a tapering dose of oral prednisone over several weeks), interferons (e.g. IFNβ-1a, and IFNβ-1b), glatiramer acetate, mitoxantrone, natalizumab, alemtuzumab, BG00012 (Biogen), cladribine, dirucotide (MBP8298), fingolimod, laquinimod, rituximab, teriflunomide, ATL1102 (Teva and Antisense Therapeutics), CDP323 (Biogen), daclizumab, estradiol, inosine, neurovax, tovaxin, mycophenolate mofetil, antimetabolites (e.g. methotrexate), macrolides/IL-2 inhibitors (e.g. FK-506), thalidomide, mitoxantrone, serotonin selective reuptake inhibitors, neuroprotectants (e.g. lithium), or combinations thereof.

### EXAMPLES

### Example 1

### Mouse Model for Treatment with HiCy and Glatiramer Acetate (HiGA)

The goal of this study is to ameliorate chronic relapsing experimental autoimmune encephalomyelitis (R-EAE) in SJL/J mice as model of relapsing/remitting Multiple Sclerosis (RRMS) with immunoablative doses of cyclophosphamide (CPA) in combination with immunization against MS reactivation via administration of glatiramer acetate.

EAE is a well-established non-primate animal model for MS. R-EAE is induced by immunization of susceptible mouse strains (SJL/J) with modified myelin proteolipid protein (PLP) 139-151 peptide (HSLGKWLGHPDKF). R-EAE takes approximately takes 2-3 weeks to develop. The genetic influence in experimental outcomes can be kept to a minimum by using the previously indicated inbred rodent strain. This would allow us to demonstrate that the obtained experimental results are due to inclusion in designated treatment groups, and observed differences between individuals are due to the disease and/or environment influences.

Treatment groups will consist of 10 mice. We anticipate having experiments which consist of 3-5 treatment groups which will involve the distinct drugs (outlined within the protocol) and combinations of these drugs (and vehicle controls), variations in drug dosages, and variations in timing of drug administration.

### R-EAE Induction and Analysis

Female SJL/J mice between 6-12 weeks old will receive one subcutaneous 100 microliter injection with 100 micrograms of modified myelin proteolipid protein (PLP) 139-151 peptide (HSLGKWLGHPDKF) that is emulsified in Freund's Incomplete Adjuvant containing Mycobacterium tuberculisis H37 Ra (CFA). CFA is an oil mixture composed of Freund's Incomplete Adjuvant mixed with heat-killed Mycobacterium tuberculosis; it is the only adjuvant known to cause this disease in this mouse strain. These injections will be done without anesthetic. For the subcutaneous adjuvant injection, the animal is held by the loose skin at the nape of its neck and injected with a 25 gauge needle in the thigh area.

Mice will be weighed and observed for clinical signs for 2 months. Clinical signs of EAE will be assessed according to the following scale: 0=no clinical disease; 1=loss of tail tonicity; 2=mild hind leg paresis; 3=moderate hind leg paralysis; 4=complete paraplegia; and 5=quadriplegia, moribund state or death. Additional behavioral outcomes may be monitored including: the animals' ability to lift their tail while walking or when touched, their ability to move all four limbs and walk in a coordinated way along the rungs of the cage lid, and overall activity level and exploratory tendencies.

EAE in the SJL/J mouse strain is expected to be an escalating type of paralysis where symptoms are preceded by obvious weight loss and mild paresis of the tail by day 10. At later time points, disease progression involves hind limb paralysis, which constitutes the effector phase, first attack of the R-EAE disease phenotype. By day 20, most mice enter the remission phase, regain weight and paralysis is abated. Mice develop a second round of EAE (relapse) at approximately 3 weeks post PLP-immunization and mice will continue to experience additional relapses and recovery phases.

Sensory function will also be tested in some experiments using a thermal sensory test. This involves determining whether there is sensation in the extremities by setting the mouse on a platform that heats up; although the heat is not extreme to the point of injury, the expected response is that the mouse should lift and lick its paw.

### Administration of Combination Therapy

Cyclophosphamide (CPA) will be administered via intraperitoneal injection in phosphate-buffered saline (PBS) (20 mg/ml) at a does of 100-300 mg/kg. CPA will be administered once to each study animal at specific time points prior to and after the effector phase of R-EAE.

Glatiramer acetate is composed of the amino acids L-alanine, L-lysine, L-glutamic acid and L-tyrosine in specified ratios and was designed to mimic one of the major myelin auto antigens involved in the induction of EAE. On day 11 (+/−2, as determined by EAE progression), glatiramer acetate will be administered subcutaneously at a dose of 50-500 micrograms/mouse in PBS/mannitol for up to five consecutive days.

For the intravenous injections, the mice are warmed while in their cage with a heat lamp (approximately 18-25 inches from the cage floor) for 3-5 minutes to dilate their blood vessels; they are then individually restrained in a cone or Broome-type restraining device (VWR catalogue number 10718-030) for the intravenous injection administered in to the lateral tail vein with a 28-30 gauge needle. Light isoflurane anesthesia will be used if animals appear distressed.

Food will be placed on the cage floor to help injured animals reach food easily. The automatic watering system is lower to the cage floor than a water bottle would be; however, if sick animals don't appear to have the strength to operate the water dispensing switch, a water bottle (and possibly direct feeding of water to individual animals at the time of their daily weigh-ins) and/or ‘hydrogel’ will be made available to the animals in those cages.

### Anticipated Results

1. Relapsing-Remitting form of EAE (RR-EAE) will be induced in SJL/J mice actively immunized with PLP 139-151(S) or following adoptive transfer of PLP 139-151(S) specific T cells.

2. Treatment of RR-EAE with 200 mg/kg CPA will result in a cessation of EAE activity in 95% of animals.

3. We anticipate a 30% spontaneous relapse rate of RR-EAE following CPA treatment with a 70% rate if induced relapse with PLP reimmunization.

4. Treatment of CPA-treated RR-EAE animals with glatiramer acetate (up to 2 mg/mouse/day) beginning 30 days after CPA treatment will not adversely affect the rate of cessation (95%) of EAE activity by CPA.

5. Treatment of CPA-treated RR-EAE with glatiramer acetate (up to 2 mg/mouse/day) starting 30 days after CPA treatment will reduce the spontaneous and induced relapse rates to 2% and 10% respectively.

### Example 2

### Mouse Model for Treatment with HiCy and ATG (HiCAT)

The goal of this study is to ameliorate chronic relapsing experimental autoimmune encephalomyelitis (R-EAE) in SJL/J mice as model of relapsing/remitting Multiple Sclerosis (RRMS) with immunoablative doses of cyclophosphamide (CPA) in combination with T-cell depleting therapies.

EAE is a well-established non-primate animal model for MS. R-EAE is induced by immunization of susceptible mouse strains (SJL/J) with modified myelin proteolipid protein (PLP) 139-151 peptide (HSLGKWLGHPDKF). R-EAE takes approximately takes 2-3 weeks to develop. The genetic influence in experimental outcomes can be kept to a minimum by using the previously indicated inbred rodent strain. This would allow us to demonstrate that the obtained experimental results are due to inclusion in designated treatment groups, and observed differences between individuals are due to the disease and/or environment influences.

Treatment groups will consist of 10 mice. We anticipate having experiments which consist of 3-5 treatment groups which will involve the distinct drugs (outlined within the protocol) and combinations of these drugs (and vehicle controls), variations in drug dosages, and variations in timing of drug administration.

### R-EAE Induction and Analysis

Female SJL/J mice between 6-12 weeks old will receive one subcutaneous 100 microliter injection with 100 micrograms of modified myelin proteolipid protein (PLP) 139-151 peptide (HSLGKWLGHPDKF) that is emulsified in Freund's Incomplete Adjuvant containing Mycobacterium tuberculisis H37 Ra (CFA). CFA is an oil mixture composed of Freund's Incomplete Adjuvant mixed with heat-killed Mycobacterium tuberculosis; it is the only adjuvant known to cause this disease in this mouse strain. These injections will be done without anesthetic. For the subcutaneous adjuvant injection, the animal is held by the loose skin at the nape of its neck and injected with a 25 gauge needle in the thigh area.

Mice will be weighed and observed for clinical signs for 2 months. Clinical signs of EAE will be assessed according to the following scale: 0=no clinical disease; 1=loss of tail tonicity; 2=mild hind leg paresis; 3=moderate hind leg paralysis; 4=complete paraplegia; and 5=quadriplegia, moribund state or death. Additional behavioral outcomes may be monitored including: the animals' ability to lift their tail while walking or when touched, their ability to move all four limbs and walk in a coordinated way along the rungs of the cage lid, and overall activity level and exploratory tendencies.

EAE in the SJL/J mouse strain is expected to be an escalating type of paralysis where symptoms are preceded by obvious weight loss and mild paresis of the tail by day 10. At later time points, disease progression involves hind limb paralysis, which constitutes the effector phase, first attack of the R-EAE disease phenotype. By day 20, most mice enter the remission phase, regain weight and paralysis is abated. Mice develop a second round of EAE (relapse) at approximately 3 weeks post PLP-immunization and mice will continue to experience additional relapses and recovery phases.

Sensory function will also be tested in some experiments using a thermal sensory test. This involves determining whether there is sensation in the extremities by setting the mouse on a platform that heats up; although the heat is not extreme to the point of injury, the expected response is that the mouse should lift and lick its paw.

### Administration of Combination Therapy

CPA will be administered intravenously in phosphate-buffered saline (PBS) (20 mg/ml) at a does of 200 mg/kg. CPA will be administered once to each study animal at specific time points prior to and after the effector phase of R-EAE. Antithymocyte antibodies raised against whole T lymphocytes will be administered intravenously concurrently with CPA at doses ranging from 12.5 to 25 micrograms per animal.

### Anticipated Results

1. Relapsing-Remitting form of EAE (RR-EAE) will be induced in SJL/J mice actively immunized with PLP 139-151(S) or following adoptive transfer of PLP 139-151(S) specific T cells.

2. Treatment of RR-EAE with 200 mg/kg CPA plus Antithymocyte antibody will result in a cessation of EAE activity in 95% of animals.

3. Over time, fewer than 10 percent of animals will show reactivation of immune system-mediated CNS inflammation and injury.

### Example 3

### Clinical Trial of HiGa in Human Diagnosed with Multiple Sclerosis

### Primary Objective

To determine if treatment with high dose cyclophosphamide (50 mg/kg IV each day for four consecutive days) or high dose cyclophosphamide and double dose glatiramer acetate (40 mg) halts or reverses the clinical progression of MS compared to low dose cyclophosphamide (1000 mg/m²) and double dose glatiramer acetate as defined by decrease in EDSS at 12 months.

### Secondary Objectives

To determine if treatment with high dose cyclophosphamide OR high dose cyclophosphamide and double dose (40 mg) glatiramer acetate causes a sustained remission (≧3 months) of MS disease activity compared to low dose cyclophosphamide and double dose glatiramer acetate at 12 months as defined by no new enhancing lesions by MRI and no new relapses (defined as the appearance of new neurologic symptoms lasting at least 48 hours and confirmed by exam).

To demonstrate superiority of high dose cyclophosphamide and double dose glatiramer acetate in the duration of sustained remission of MS disease activity compared to high dose cyclophosphamide alone at 24 months in the proportion of relapse free individuals defined by the appearance of new neurologic symptoms lasting at least 48 hours and confirmed by examination during the 24 months of the study OR new enhancing lesions on MRI at 24 months.

To evaluate the safety and tolerability of high dose cyclophosphamide and double dose glatiramer acetate in individuals with RRMS treated for up to 24 months.

### Study Design

We propose a 12-month, randomized, multi-center, rater-blinded (pre and post) trial in approximately 222 individuals with aggressive relapsing remitting MS (RRMS) with a follow up extension of 12 months. Approximately 222 individuals will be randomized to one of the following three treatment arms (74 in each arm):
a. low dose cyclophosphamide and double dose glatiramer acetate.
b. high dose cyclophosphamide; and
c. high dose cyclophosphamide and double dose glatiramer acetate.

### Inclusion Criteria:

Individuals, male or female, meeting of following criteria may be enrolled in the clinical trial:
a. between the ages of 18 and 50 years;
b. a diagnosis of clinically definite relapsing-remitting MS according to the McDonald Criteria;
c. two (2) or more total gadolinium enhancing lesions on a brain and/or spinal cord MRI at screening;
d. at least one clinical relapse in the last year;
e. an EDSS ranging from 1.5 to 6.5 inclusive; individuals with EDSS ≧5.5 should have been sustained at that disability for ≦3 months;
f. a sustained (≧3 months) increase of >1.0 on the EDSS (historical estimate allowed) between 1.5 and 5.5 or >0.5 between 5.5 and 6.5 in the preceding year;
g. written informed consent prior to any testing under this protocol, including screening tests and evaluations that are not considered part of the individual's routine care; and
h. for females, a negative pregnancy test prior to entry into the study.

### Exclusion Criteria

The following individuals will be excluded from the clinical trial:
a. any individual at risk of pregnancy;
b. any individual exhibiting cardiac ejection fraction of <45%;
c. any individual exhibiting serum creatinine levels >2.0;
d. any individual who is pre-terminal or moribund;
e. any individual exhibiting bilirubin levels >2.0, and/or transaminases levels >2× normal;
f. any individual with pacemakers and implants who cannot get serial MRIs;
g. any individual with active infections until infection is resolved; or
h. any individual with WBC count <3000 cells/μl; platelets <100,000 cells/μl; and untransfused hemoglobin <10 g/dl.
   Removal of Individuals from the Study

Individuals may withdraw from the study at any time for any reason. Any investigator may discontinue a individual for any of the following reasons:
a. the individual experiences a medical emergency that necessitates discontinuation of therapy during the high dose cyclophosphamide treatment in the hospital;
b. the individual experiences a serious adverse event that is judged to be likely related to high dose cyclophosphamide and/or is of severity that warrants discontinuation of high dose cyclophosphamide during hospital stay; and
c. for any medical reason at the discretion of the investigator.

### High Dose Cyclophosphamide Administration

The high dose cyclophosphamide treatment will be performed under the supervision of Oncology physicians and staff.

Individuals will receive high dose cyclophosphamide intravenously on Day −3 to Day 0. The dose of high dose cyclophosphamide will be calculated according to ideal body weight. Ideal body weight will be determined according to the current policy used in the Bone Marrow Transplant program. If the individual's actual weight is less than ideal, the actual weight will be used to calculate the dose of cyclophosphamide. Individuals are scheduled to receive only one course of therapy.

Adequate diuresis should be maintained before and following high dose cyclophosphamide administration to prevent hemorrhagic cystitis. Prophylaxis for cyclophosphamide induced hemorrhagic cystitis (generally either MESNA or forced diuresis) will be directed according to established clinical practice guidelines used by the SCT program.

On Day 6 (six days after completion of high dose cyclophosphamide) individuals will receive granulocyte colony stimulating factor (5 μg/kg/d) until the absolute neutrophil count exceeds 1.0×10⁹ per liter for two consecutive days. Individuals are also routinely give antibiotics (norfloxacin, fluconazole and valacyclovir) until the return of normal neutrophil counts.

### Low Dose Cyclophosphamide Administration

Low dose cyclophosphamide will be administered at 1000 mg/m² IV in 100 cc NSS over two hours. Prehydration will consist of 2L NSS over 4 hours and post-hydration will consist of 2L NSS over 4 hours. Dose will be calculated according to ideal body weight as above.

### Glatiramer Acetate Administration

Double dose glatiramer acetate will be administered daily subcutaneously beginning at 30 days after the last dose of high dose cyclophosphamide (Day 0) or the single lower dose cyclophosphamide injection.

### Post Treatment Discharge

Individuals will be hospitalized for a minimum of 4 days as clinically indicated. They will then be admitted to an outindividual care facility until return of neutrophil count as per standard protocols (usually 2-3 weeks after the last dose of high dose cyclophosphamide).

### MRI Evaluations

MRI evaluations are conducted at months −3, 0, 3, 6, 9, 12, 15, 18, 21 and 24 after treatment. These will enable the understanding of the course of the disease progression after treatment. The mean number of gadolinium enhancing lesions will be monitored to assess the change in disease activity. Change from baseline (average number of gad-enhancing lesions at months −3 and 0) to follow-up (average number of gad-enhancing lesions at months 15 and 18) will be assessed. Further, serial MRIs at months 3, 6, 9 and 12 months would enable an understanding of the change in disease activity through 2 years, while also monitoring safety of High dose cyclophosphamide. Other parameters—T2 lesion load and brain parenchymal fraction are also measures of disease activity that correlate with accrual of disability and changes will be assessed through the length of the study. Scans will be performed on a 1.5 Tesla General Electric scanner (Milwaukee Wis.) with echo speed or twin speed gradients.

MRI criteria for disease progression:
a. number of gadolinium enhancing lesions;
b. T2 lesion load; and
c. brain parenchymal fraction.

Analysis of MRI scans:
a. Contrast-enhancing lesions will be counted from the axial 3 mm contiguous slices with verification on the coronal images. If a lesion is seen on one sequence but not the other, it will be counted as an enhancing plaque if it is also seen on a long TR pulse sequence. Total disease burden will be determined from scans from the cervicomedullary junction to the vertex based on the number of enhancing plaques.
b. The volume of multiple sclerosis plaques will be determined from analysis of the FLAIR scans as they provide the maximal contrast to noise between MS plaques and underlying cerebrospinal fluid (CSF) versus normal white and gray matter. However in the event of cystic MS plaques which would have dark signal on FLAIR scans, we will utilize the proton density-T2-weighted pulse sequences to identify these lesions and supplement the FLAIR volume assessment with these additional MS plaques. Thresholding and 3D volumetric analysis will be performed using computer-assisted volumetry.
c. Total brain parenchymal volume will be performed suing standard stripping algorithms to remove the skull and overlying soft tissue. Using thresholding and manual corrections, the CSF will then be removed to allow an analysis of brain parenchyma volume.
d. Two radiologists will read the MRI scans independently. If there is greater than 10% discrepancy between interpretations, a third radiologist will be asked to interpret the MRI scans. The reported interpretation will be the average of the three readings (on T2 plaque volume and brain parenchymal fraction) or will reflect the two interpretations in agreement (for the number of enhancing lesions). The data will be recorded on the CRFs and input into the database.

### Neurological/Clinical Evaluation

Neurological exam will also be conducted at baseline and every 3 months after the high dose cyclophosphamide treatment for the duration of the study (24 months). To determine the course of the disease, the clinical measures used are the Multiple Sclerosis Functional Composite (MSFC) and the Expanded Disability Status Scale (EDSS). A research nurse/coordinator will be trained to administer the MSFC and a study neurologist will examine the individual to provide an EDSS score.

The EDSS ranges from 0 (normal) to 10 (death due to MS), based on neurological examination of eight functional systems (visual, brainstem, sensory, cerebellar, sphincter, cerebral and others).

The MSFC is designed to test gait, upper extremity dexterity and cognition. The three subtests are (a) 25 foot timed walk (25TW); (b) 9-hole peg test (9-HPT); and (c) Paced Auditory Serial Addition Test (PASAT-3). The PASAT test requires individuals to add consecutive numbers as they are presented on an auditory tape and respond orally with the accurate sum. As each digit is presented, the individual must sum that number with the digit that was presented prior to it rather than with the individual's previous response.

### Example 4

### Clinical Trial of HiCAT in Human Diagnosed with Multiple Sclerosis

### Primary Objective

To determine if treatment with high dose cyclophosphamide (50 mg/kg IV each day for four consecutive days) and antithymocyte globulin (2.5 μg/kg/day) halts or reverses the clinical progression of MS compared to high dose cyclophosphamide alone as defined by decrease in EDSS at 12 months.

### Secondary Objectives

To determine if treatment with high dose cyclophosphamide and antithymocyte globulin causes a sustained remission (≧3 months) of MS disease activity compared to high dose cyclophosphamide at 12 months as defined by no new enhancing lesions by MRI and no new relapses (defined as the appearance of new neurologic symptoms lasting at least 48 hours and confirmed by exam).

To demonstrate superiority of high dose cyclophosphamide and antithymocyte globulin in the duration of sustained remission of MS disease activity compared to high dose cyclophosphamide alone at 24 months in the proportion of relapse free individuals as defined by the appearance of new neurologic symptoms lasting at least 48 hours and confirmed by examination during the 24 months of the study OR new enhancing lesions on MRI at 24 months.

To evaluate the safety and tolerability high dose cyclophosphamide and antithymocyte globulin in individuals with RRMS treated for up to 24 months.

### Study Design

We propose a 12-month, randomized, multi-center, rater-blinded (pre and post) trial in approximately 222 individuals with aggressive relapsing remitting MS (RRMS) with a follow up extension of 12 months. Approximately 222 individuals will be randomized to one of the following two treatment arms (111 in each arm):
a. high dose cyclophosphamide; and
b. high dose cyclophosphamide and antithymocyte globulin.

### Inclusion Criteria:

Individuals, male or female, meeting all of following criteria may be enrolled in the clinical trial:
a. between the ages of 18 and 50 years;
b. a diagnosis of clinically definite relapsing-remitting MS according to the McDonald Criteria;
c. two (2) or more total gadolinium enhancing lesions on a brain and/or spinal cord MRI at screening;
d. at least one clinical relapse in the last year;
e. an EDSS ranging from 1.5 to 6.5 inclusive; individuals with EDSS ≧5.5 should have been sustained at that disability for ≦3 months;
f. a sustained (≧3 months) increase of >1.0 on the EDSS (historical estimate allowed) between 1.5 and 5.5 or >0.5 between 5.5 and 6.5 in the preceding year;
g. written informed consent prior to any testing under this protocol, including screening tests and evaluations that are not considered part of the individual's routine care; and
h. for females, a negative pregnancy test prior to entry into the study.

### Exclusion Criteria

The following individuals will be excluded from the clinical trial:
a. any individual at risk of pregnancy;
b. any individual exhibiting cardiac ejection fraction of <45%;
c. any individual exhibiting serum creatinine levels >2.0;
d. any individual who is pre-terminal or moribund;
e. any individual exhibiting bilirubin levels >2.0, and/or transaminases levels >2× normal;
f. any individual with pacemakers and implants who cannot get serial MRIs;
g. any individual with active infections until infection is resolved; or
h. any individual with WBC count <3000 cells/μl; platelets <100,000 cells/μl; and untransfused hemoglobin <10 g/dl.
   Removal of Individuals from the Study

Individuals may withdraw from the study at any time for any reason. Any investigator may discontinue a individual for any of the following reasons:
a. the individual experiences a medical emergency that necessitates discontinuation of therapy during the high dose cyclophosphamide treatment in the hospital;
b. the individual experiences a serious adverse event that is judged to be likely related to high dose cyclophosphamide and/or is of severity that warrants discontinuation of high dose cyclophosphamide during hospital stay; and
c. for any medical reason at the discretion of the investigator.

### High Dose Cyclophosphamide Administration

The high dose cyclophosphamide treatment will be performed under the supervision of oncology physicians and staff.

Individuals will receive high dose cyclophosphamide 50 mg/kg/d intravenously on Day −3 to Day 0. The dose of high dose cyclophosphamide will be calculated according to ideal body weight. Ideal body weight will be determined according to the current policy used in the Bone Marrow Transplant program. If the individual's actual weight is less than ideal, the actual weight will be used to calculate the dose of cyclophosphamide. Individuals are scheduled to receive only one course of therapy.

Adequate diuresis should be maintained before and following high dose cyclophosphamide administration to prevent hemorrhagic cystitis. Prophylaxis for cyclophosphamide induced hemorrhagic cystitis (generally either MESNA or forced diuresis) will be directed according to established clinical practice guidelines used by the SCT program.

On Day 6 (six days after completion of high dose cyclophosphamide) individuals will receive granulocyte colony stimulating factor (5 μg/kg/d) until the absolute neutrophil count exceeds 1.0×10⁹ per liter for two consecutive days. Individuals are also routinely give antibiotics (norfloxacin, fluconazole and valacyclovir) until the return of normal neutrophil counts.

### Antithymocyte Globulin Administration

Antithymocyte globulin will be administered daily by IV concurrently with high dose cyclophosphamide or the single lower dose cyclophosphamide injection.

### Post Treatment Discharge

Individuals will be hospitalized for a minimum of 4 days as clinically indicated. They will then be admitted to an outindividual care facility until return of neutrophil count as per standard protocols (usually 2-3 weeks after the last dose of high dose cyclophosphamide).

### MRI Evaluations

MRI evaluations are conducted at months −3, 0, 3, 6, 9, 12, 15, 18, 21 and 24 after treatment. These will enable the understanding of the course of the disease progression after treatment. The mean number of gadolinium enhancing lesions will be monitored to assess the change in disease activity. Change from baseline (average number of gad-enhancing lesions at months −3 and 0) to follow-up (average number of gad-enhancing lesions at months 15 and 18) will be assessed. Further, serial MRIs at months 3, 6, 9 and 12 months would enable an understanding of the change in disease activity through 2 years, while also monitoring safety of high dose cyclophosphamide. Other parameters—T2 lesion load and brain parenchymal fraction are also measures of disease activity that correlate with accrual of disability and changes will be assessed through the length of the study. Scans will be performed on a 1.5 Tesla General Electric scanner (Milwaukee Wis.) with echo speed or twin speed gradients.

MRI criteria for disease progression:
a. number of gadolinium enhancing lesions;
b. T2 lesion load; and
c. brain parenchymal fraction.

Analysis of MRI scans:
a. Contrast-enhancing lesions will be counted from the axial 3 mm contiguous slices with verification on the coronal images. If a lesion is seen on one sequence but not the other, it will be counted as an enhancing plaque if it is also seen on a long TR pulse sequence. Total disease burden will be determined from scans from the cervicomedullary junction to the vertex based on the number of enhancing plaques.
b. The volume of multiple sclerosis plaques will be determined from analysis of the FLAIR scans as they provide the maximal contrast to noise between MS plaques and underlying cerebrospinal fluid (CSF) versus normal white and gray matter. However in the event of cystic MS plaques which would have dark signal on FLAIR scans, we will utilize the proton density-T2-weighted pulse sequences to identify these lesions and supplement the FLAIR volume assessment with these additional MS plaques. Thresholding and 3D volumetric analysis will be performed using computer-assisted volumetry.
c. Total brain parenchymal volume will be performed suing standard stripping algorithms to remove the skull and overlying soft tissue. Using thresholding and manual corrections, the CSF will then be removed to allow an analysis of brain parenchyma volume.
d. Two radiologists will read the MRI scans independently. If there is greater than 10% discrepancy between interpretations, a third radiologist will be asked to interpret the MRI scans. The reported interpretation will be the average of the three readings (on T2 plaque volume and brain parenchymal fraction) or will reflect the two interpretations in agreement (for the number of enhancing lesions). The data will be recorded on the CRFs and input into the database.

### Neurological/Clinical Evaluation

Neurological exam will also be conducted at baseline and every 3 months after the high dose cyclophosphamide treatment for the duration of the study (24 months). To determine the course of the disease, the clinical measures used are the Multiple Sclerosis Functional Composite (MSFC) and the Expanded Disability Status Scale (EDSS). A research nurse/coordinator will be trained to administer the MSFC and a study neurologist will examine the individual to provide an EDSS score.

The EDSS ranges from 0 (normal) to 10 (death due to MS), based on neurological examination of eight functional systems (visual, brainstem, sensory, cerebellar, sphincter, cerebral and others).

The MSFC is designed to test gait, upper extremity dexterity and cognition. The three subtests are (a) 25 foot timed walk (25TW); (b) 9-hole peg test (9-HPT); and (c) Paced Auditory Serial Addition Test (PASAT-3). The PASAT test requires individuals to add consecutive numbers as they are presented on an auditory tape and respond orally with the accurate sum. As each digit is presented, the individual must sum that number with the digit that was presented prior to it rather than with the individual's previous response.

### Example 5

### Clinical Trial of HiCAT and Glatiramer Acetate in Human Diagnosed with Multiple Sclerosis

### Primary Objective

To determine if treatment with high dose cyclophosphamide (50 mg/kg IV each day for four consecutive days), antithymocyte globulin (2.5 μg/kg/day), and double dose glatiramer acetate (40 mg) halts or reverses the clinical progression of MS compared to high dose cyclophosphamide alone as defined by decrease in EDSS at 12 months.

### Secondary Objectives

To determine if treatment with high dose cyclophosphamide, antithymocyte globulin, and double dose glatiramer acetate causes a sustained remission (≧3 months) of MS disease activity compared to high dose cyclophosphamide alone at 12 months as defined by no new enhancing lesions by MRI and no new relapses (defined as the appearance of new neurologic symptoms lasting at least 48 hours and confirmed by exam).

To demonstrate superiority of high dose cyclophosphamide, antithymocyte globulin, and glatiramer acetate in the duration of sustained remission of MS disease activity compared to high dose cyclophosphamide alone at 24 months in the proportion of relapse free individuals as defined by the appearance of new neurologic symptoms lasting at least 48 hours and confirmed by examination during the 24 months of the study OR new enhancing lesions on MRI at 24 months.

To evaluate the safety and tolerability of high dose cyclophosphamide, antithymocyte globulin, and glatiramer acetate in individuals with RRMS treated for up to 24 months.

### Study Design

We propose a 12-month, randomized, multi-center, rater-blinded (pre and post) trial in approximately 222 individuals with aggressive relapsing remitting MS (RRMS) with a follow up extension of 12 months. Approximately 222 individuals will be randomized to one of the following two treatment arms (111 in each arm):
a. high dose cyclophosphamide; and
b. high dose cyclophosphamide, antithymocyte globulin, and glatiramer acetate.

### Inclusion Criteria:

Individuals, male or female, meeting all of following criteria may be enrolled in the clinical trial:
a. between the ages of 18 and 50 years;
b. a diagnosis of clinically definite relapsing-remitting MS according to the McDonald Criteria;
c. two (2) or more total gadolinium enhancing lesions on a brain and/or spinal cord MRI at screening;
d. at least one clinical relapse in the last year;
e. an EDSS ranging from 1.5 to 6.5 inclusive; individuals with EDSS ≧5.5 should have been sustained at that disability for ≦3 months;
f. a sustained (≧3 months) increase of >1.0 on the EDSS (historical estimate allowed) between 1.5 and 5.5 or >0.5 between 5.5 and 6.5 in the preceding year;
g. written informed consent prior to any testing under this protocol, including screening tests and evaluations that are not considered part of the individual's routine care; and
h. for females, a negative pregnancy test prior to entry into the study.

### Exclusion Criteria

The following individuals will be excluded from the clinical trial:
a. any individual at risk of pregnancy;
b. any individual exhibiting cardiac ejection fraction of <45%;
c. any individual exhibiting serum creatinine levels >2.0;
d. any individual who is pre-terminal or moribund;
e. any individual exhibiting bilirubin levels >2.0, and/or transaminases levels >2× normal;
f. any individual with pacemakers and implants who cannot get serial MRIs;
g. any individual with active infections until infection is resolved; or
h. any individual with WBC count <3000 cells/μl; platelets <100,000 cells/μl; and untransfused hemoglobin <10 g/dl.
   Removal of Individuals from the Study

Individuals may withdraw from the study at any time for any reason. Any investigator may discontinue a individual for any of the following reasons:
a. the individual experiences a medical emergency that necessitates discontinuation of therapy during the high dose cyclophosphamide treatment in the hospital;
b. the individual experiences a serious adverse event that is judged to be likely related to high dose cyclophosphamide and/or is of severity that warrants discontinuation of high dose cyclophosphamide during hospital stay; and
c. for any medical reason at the discretion of the investigator.

### High Dose Cyclophosphamide Administration

The high dose cyclophosphamide treatment will be performed under the supervision of oncology physicians and staff.

Individuals will receive high dose cyclophosphamide 50 mg/kg/d intravenously on Day −3 to Day 0. The dose of high dose cyclophosphamide will be calculated according to ideal body weight. Ideal body weight will be determined according to the current policy used in the Bone Marrow Transplant program. If the individual's actual weight is less than ideal, the actual weight will be used to calculate the dose of cyclophosphamide. Individuals are scheduled to receive only one course of therapy.

Adequate diuresis should be maintained before and following high dose cyclophosphamide administration to prevent hemorrhagic cystitis. Prophylaxis for cyclophosphamide induced hemorrhagic cystitis (generally either MESNA or forced diuresis) will be directed according to established clinical practice guidelines used by the SCT program.

On Day 6 (six days after completion of high dose cyclophosphamide) individuals will receive granulocyte colony stimulating factor (5 μg/kg/d) until the absolute neutrophil count exceeds 1.0×10⁹ per liter for two consecutive days. Individuals are also routinely give antibiotics (norfloxacin, fluconazole and valacyclovir) until the return of normal neutrophil counts.

### Antithymocyte Globulin Administration

Antithymocyte globulin will be administered daily by IV concurrently with high dose cyclophosphamide or the single lower dose cyclophosphamide injection.

### Glatiramer Acetate Administration

Double dose glatiramer acetate will be administered daily subcutaneously beginning at 30 days after the last dose of high dose cyclophosphamide (Day 0) or the single lower dose cyclophosphamide injection.

### Post Treatment Discharge

Individuals will be hospitalized for a minimum of 4 days as clinically indicated. They will then be admitted to an outindividual care facility until return of neutrophil count as per standard protocols (usually 2-3 weeks after the last dose of high dose cyclophosphamide).

### MRI Evaluations

MRI evaluations are conducted at months −3, 0, 3, 6, 9, 12, 15, 18, 21 and 24 after treatment. These will enable the understanding of the course of the disease progression after treatment. The mean number of gadolinium enhancing lesions will be monitored to assess the change in disease activity. Change from baseline (average number of gad-enhancing lesions at months −3 and 0) to follow-up (average number of gad-enhancing lesions at months 15 and 18) will be assessed. Further, serial MRIs at months 3, 6, 9 and 12 months would enable an understanding of the change in disease activity through 2 years, while also monitoring safety of high dose cyclophosphamide. Other parameters—T2 lesion load and brain parenchymal fraction are also measures of disease activity that correlate with accrual of disability and changes will be assessed through the length of the study. Scans will be performed on a 1.5 Tesla General Electric scanner (Milwaukee Wis.) with echo speed or twin speed gradients.

MRI criteria for disease progression:
a. number of gadolinium enhancing lesions;
b. T2 lesion load; and
c. brain parenchymal fraction.

Analysis of MRI scans:
a. Contrast-enhancing lesions will be counted from the axial 3 mm contiguous slices with verification on the coronal images. If a lesion is seen on one sequence but not the other, it will be counted as an enhancing plaque if it is also seen on a long TR pulse sequence. Total disease burden will be determined from scans from the cervicomedullary junction to the vertex based on the number of enhancing plaques.
b. The volume of multiple sclerosis plaques will be determined from analysis of the FLAIR scans as they provide the maximal contrast to noise between MS plaques and underlying cerebrospinal fluid (CSF) versus normal white and gray matter. However in the event of cystic MS plaques which would have dark signal on FLAIR scans, we will utilize the proton density-T2-weighted pulse sequences to identify these lesions and supplement the FLAIR volume assessment with these additional MS plaques. Thresholding and 3D volumetric analysis will be performed using computer-assisted volumetry.
c. Total brain parenchymal volume will be performed suing standard stripping algorithms to remove the skull and overlying soft tissue. Using thresholding and manual corrections, the CSF will then be removed to allow an analysis of brain parenchyma volume.
d. Two radiologists will read the MRI scans independently. If there is greater than 10% discrepancy between interpretations, a third radiologist will be asked to interpret the MRI scans. The reported interpretation will be the average of the three readings (on T2 plaque volume and brain parenchymal fraction) or will reflect the two interpretations in agreement (for the number of enhancing lesions). The data will be recorded on the CRFs and input into the database.

### Neurological/Clinical Evaluation

Neurological exam will also be conducted at baseline and every 3 months after the high dose cyclophosphamide treatment for the duration of the study (24 months). To determine the course of the disease, the clinical measures used are the Multiple Sclerosis Functional Composite (MSFC) and the Expanded Disability Status Scale (EDSS). A research nurse/coordinator will be trained to administer the MSFC and a study neurologist will examine the individual to provide an EDSS score.

The EDSS ranges from 0 (normal) to 10 (death due to MS), based on neurological examination of eight functional systems (visual, brainstem, sensory, cerebellar, sphincter, cerebral and others).

The MSFC is designed to test gait, upper extremity dexterity and cognition. The three subtests are (a) 25 foot timed walk (25TW); (b) 9-hole peg test (9-HPT); and (c) Paced Auditory Serial Addition Test (PASAT-3). The PASAT test requires individuals to add consecutive numbers as they are presented on an auditory tape and respond orally with the accurate sum. As each digit is presented, the individual must sum that number with the digit that was presented prior to it rather than with the individual's previous response.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

All documents mentioned herein are incorporated herein by reference in their entirety.

1. A method of treating neurological autoimmune disorders, comprising administering to an individual in need having an aldehyde dehydrogenase level in the CD 4+ T cells less than a predetermined threshold:
(a) about 10 to about 70 mg/kg/day of cyclophosphamide;
(b) about 1 to about 10 μg/kg/day of granulocyte colony stimulating factor; and
(c) about 10 mg/day to about 80 mg/day of glatiramer acetate.

2. The method of claim 1, further comprising determining the level of aldehyde dehydrogenase in the individual's CD 4+ T cells.

3. The method of claim 2, further comprising monitoring the level of aldehyde dehydrogenase in the individual's CD 4+ T cells.

4. The method of claim 1, wherein at least about 50 mg/kg/day of cyclophosphamide is administered to the individual.

5. The method of claim 1, wherein at least about 5 μg/kg/day of granulocyte colony stimulating factor is administered to the individual.

6. The method of claim 1, wherein at least about 40 mg/day of glatiramer acetate is administered to the individual.

7-24. (canceled)

25. A method of treating neurological autoimmune disorders, comprising administering to an individual in need having an aldehyde dehydrogenase level in the CD 4+ T cells less than a predetermined threshold:
(a) about 10 to about 70 mg/kg/day of cyclophosphamide;
(b) up to about 5 mg/kg/day of antithymocyte globulin; and
(c) about 1 to about 10 μg/kg/day of granulocyte colony stimulating factor.

26. The method of claim 25, further comprising determining the level of aldehyde dehydrogenase in the individual's CD 4+ T cells.

27. The method of claim 26, further comprising monitoring the level of aldehyde dehydrogenase in the individual's CD 4+ T cells.

28-48. (canceled)

49. A method of selecting an individual for treatment with cyclophosphamide comprising selecting an individual for treatment if an aldehyde dehydrogenase level in a biological sample from the individual exceeds a predetermined threshold; or selecting an alternative treatment if the aldehyde dehydrogenase level observed in the biological sample is below a predetermined threshold.

50. The method of claim 49, wherein the biological sample is blood, and/or white blood cells.

51. The method of claim 50, wherein the white blood cells are T cells.

52. The method of claim 51, wherein the T cells are CD 4+ T cells.

53. The method of claim 49, wherein aldehyde dehydrogenase level is determined by a fluorescent aldehyde dehydrogenase substrate assay.

54. The method of claim 53, wherein the fluorescent aldehyde dehydrogenase substrate is ALDEFLUOR®.

55-92. (canceled)

93. A composition, comprising cyclophosphamide in solution, wherein the cyclophosphamide in solution has been reconstituted from lyophilized cyclophosphamide.

94. The composition of claim 93, wherein the cyclophosphamide is reconstituted in phosphate buffered saline.

95. The composition of claim 93, wherein the concentration of cyclophosphamide in the solution is at least about 20 mg/ml.

96. The composition of claim 93 for use as an immunoablative agent in an individual with an autoimmune neurological disorder.

97. The composition of claim 96, wherein the individual has an autoimmune neurological disorder selected from multiple sclerosis, Guillain-Barre syndrome, Lambert-Eaton myasthenic syndrome, myasthenia gravis, transverse myelitis, systemic lupus erythematosus (SLE or lupus), acute disseminated encephalomyelitis, autoimmune inner ear disease, narcolepsy, neuromyotonia, schizophrenia, or combinations thereof.

98. The composition of claim 97, wherein the autoimmune neurological disorder is multiple sclerosis.
